(19) United States
(12) Patent Application Publication
(10) Pub. No.: US 2023/0132012 A1
(43) Pub. Date: Apr. 27, 2023

(54) VERTICAL-VIA ROTARY VALVES, MICROBIOREACTORS AND APPLICATIONS OF SAME

(71) Applicant: VANDERBILT UNIVERSITY, Nashville, TN (US)

(72) Inventors: David K. Schaffer, Nashville, TN (US); John P. Wikswo, Brentwood, TN (US); Ronald S. Reiserer, Nashville, TN (US); Michael D. Geuy, Nashville, TN (US); Eric C. Spivey, Nashville, TN (US); Clayton M. Britt, Franklin, KY (US); Jacquelyn A. Brown, Nashville, TN (US); Dmitry A. Markov, Nashville, TN (US); Shannon Faley, Nashville, TN (US); Lisa J. McCawley, Nashville, TN (US); Philip C. Samson, Nashville, TN (US)

(21) Appl. No.: 17/917,963

(22) PCT Filed: Apr. 30, 2021
(86) PCT No.: PCT/US2021/030219

§ 371 Date: Oct. 10, 2022

Related U.S. Application Data
(60) Provisional application No. 63/017,744, filed on Apr. 30, 2020.

(51) Int. Cl. B01L 3/00

(52) U.S. Cl. CPC B01L 3/502738; B01L 3/502715; B01L 2200/0605; B01L 2300/0861; B01L 2400/0655; B01L 2300/0858; B01L 2300/0816; B01L 2400/0481; B01L 2300/123; B01L 2300/0803; B01L 2400/0644

(57) ABSTRACT

One aspect of this invention relates to a vertical-via rotary valve including a valve body having a housing; one or more fluidic channels, each fluidic channel having a vertical channel portion defined in the valve body and being adjacent to the housing, wherein a fluid flow through the vertical channel portion is controllable by deforming a sidewall of the vertical channel portion; and an actuator received in the housing and rotatably engaged with the one or more fluidic channels to operably control the fluid flow through the vertical channel portion of each fluid channel.

### CROSS-REFERENCE TO RELATED PATENT APPLICATION

This application claims priority to and the benefit of U.S. Provisional Patent Application Ser. No. 63/017,744, filed Apr. 30, 2020, which is incorporated herein by reference in its entirety.

### STATEMENT AS TO RIGHTS UNDER FEDERALLY-SPONSORED RESEARCH

This invention was made with government support under Grant No. UH3TR002097 and 3UH3TR002097-04S1 awarded by the NIH National Center for Advancing Translational Sciences (NCATS), National Institute of Neurological Disorders and Stroke (NINDS), and Eunice Kennedy Shriver National Institute of Child Health and Human Development (NICHD); Grant No. U01TR002383 and (through Vanderbilt University Medical Center) UL1TR002243 awarded by NCATS; Grant No. U01CA202229 awarded by the National Cancer Institute (NCI), and Grant No. HHSN271201 700044C awarded by the National Center for Advancing Translational Sciences (NCATS) through CFD Research Corporation. The government has certain rights in the invention.

### FIELD OF THE INVENTION

The invention relates generally to microfluidic systems, and more particularly to rotary via valves, microbioreactors, and applications of the same.

### BACKGROUND INFORMATION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The need to study the mechanisms of infection and response to therapies of highly infectious pathogens, such as the eastern, western, and Venezuelan equine encephalitis alphaviruses, and the highly infective coronaviruses, such as SARS-CoV, MERS-CoV, and SARS-CoV-2, require the use of biosafety level 3 (BSL-3) or BSL-4 facilities to ensure containment of the pathogen and protection of the researchers studying them. Conventional two-dimensional (2D) monoculture of cells on the bottom of a plastic flask, Petri dish, or well plate is inadequate to recapitulate the complexity and multi-organ involvement of the diseases associated with these pathogens. For example, the Venezuelan equine encephalitis alphavirus (VEEV) might enter the brain either through the olfactory bulb via the nasal cavities, or by entering the circulation at the lungs and then crossing the blood-brain barrier. Infection with SARS-CoV-2 can result in pulmonary, central nervous system (CNS), and gastrointestinal (GI) involvement in the initial stages of the infection, a massive inflammatory response, and multi-organ failure, and the detailed sequence of events during this coupled infection/inflammatory response is poorly understood. Microphysiological systems (MPS) models that utilize individual or coupled 2D or 3D organs-on-chips or tissue chips have been shown to better recapitulate human physiology and pathology than 2D cultures on plastic, but the existing devices are ill-suited for use in BSL-3 and BSL-4 facilities, which require strict secondary containment of all experiments involving pathogens and thorough decontamination by solvent immersion, autoclaving, or other techniques of hardware or samples being removed from the BSL-3 or BSL-4 facilities. The easiest way to decontaminate disposable materials and supplies coming out of such facilities is by incineration. Hence the maintenance and repair of complex instruments within a BSL-3 or BSL-4 environment presents major technical and economic challenges.

Living cells require fresh media to provide nutrients and remove toxic waste products. In the human body, nutrients are supplied through the GI tract, and metabolites are detoxified or eliminated by the kidney, liver, gut, lungs, skin, and other organs. For in vitro studies, 2D culture on plastic and MPS are distinguished primarily by the means for media exchange: in 2D culture a substantial fraction of the media is exchanged every day or two, thereby depriving the cells of the signaling and metabolic factors that condition the media. In MPS models, continuous or repetitive perfusion provides the fresh media and removes metabolic waste products. The perfusion flow rate and volumes can be adjusted to minimize the dilution of signaling molecules and metabolites and wash away signaling factors and metabolites that should be at a low concentration in normal tissue. Hence perfusion maintains these concentrations at the appropriate levels for both physiological effect and detectability.

Many commercial and research MPS models offer impressive capabilities but require a high level of both infrastructure and personnel expertise. Some commercial MPS lung-on-a-chip, airway chip, and liver-chip assay platforms include perfusion control hardware that is large, interconnected by tubing to pressurize reservoirs pneumatically or for peristaltic pumping to perfuse the chips that cannot be easily utilized in a BSL-3 lab, expensive, and not disposable. Other platforms require external syringe pumps and specially trained personnel to run their devices, as well as pumps with pressure fit connections that could pose a leak hazard in the BSL-3 environment. Systems with tubing and pumps often require bubble traps, since small changes in the temperature of the media being pumped can lead to the release of bubbles which, having no other means of escape, can strip cells from the surfaces of bioreactors as the fluid is pumped through tubing, channels, and chambers. Tissue chip systems that use rockers for gravity perfusion are ill-suited for BSL-3 and often cannot adequately recapitulate the continuous, unidirectional flow of the in vivo environment. In the current COVID-19 crisis and to meet a high demand for effective research tools that are BSL-3 compliant, there is a clear need for an MPS chip that is both easy to make and easy to use, and that provides physiologically relevant responses to viral and pharmaceutical challenges.

Often gravity-induced flow through a microfluidic network or a bioreactor is a practical and very inexpensive alternative to using complicated and bulky pumps. For applications that do not require fast flows or repeated adjustments of the flow rate, or where the use of external pumping devices is cost or space prohibitive, gravity-induced flow offers a simple solution. The chip can contain both the supply and collection reservoirs and be tilted to provide the height difference between the two; without tilting, open, vertical pipette tips can be inserted into the fluidic channels, or, also without tilting, cylindrical glass reservoirs can be bonded to the chip to deliver fluid and store waste, with the flow rate depending upon the height difference between the supply and waste reservoirs.

Gravity perfusion has been successfully used in the past for long-term cell culture in a thick tissue bioreactor (TTB). A simple way to control the flow rate in the gravity driven system for a given pressure differential without tilting is to have control over fluidic resistance of the network. This approach was implemented in our TTB by introducing a constricting output manifold. By selecting a manifold with an appropriate channel height, one can produce a sufficient resistance to flow that, in combination with the fixed height of the supply reservoir, could be used to passively limit the flow through the bioreactor. Such an approach works well for a given fluidic configuration but lacks flexibility. An adjustable valve that can change fluidic resistance of the flow path depending on experimental requirements would be a significant improvement and a more universal solution to the passive flow regulation problem.

Therefore, a heretofore unaddressed need exists in the art to address the aforementioned deficiencies and inadequacies.

### SUMMARY OF THE INVENTION

One of the objectives of this invention is to provide a new class of gravity or pressure-perfused bioreactors that are designed specifically for use in a BSL-3 or BSL-4 environment, but have a large number of other possible applications in drug discovery, toxicology, biology, and medicine, to meet the above-noted needs.

In one aspect, the invention relates to a vertical-via rotary valve (VVRV), comprising: a valve body having a housing; one or more fluidic channels, each fluidic channel having a vertical channel portion defined in the valve body and being adjacent to the housing, wherein a fluid flow through the vertical channel portion is controllable by deforming a sidewall of the vertical channel portion; and an actuator received in the housing and rotatably engaged with the one or more fluidic channels to operably control the fluid flow through the vertical channel portion of each fluid channel.

In one embodiment, the valve body is formed of an elastomeric material including polydimethylsiloxane (PDMS).

In one embodiment, the vertical channel portion of each fluidic channel is a vertical via.

In one embodiment, the actuator features a vertical trough aligned with the vertical via, such that when the actuator un-pinches the vertical via and the fluid flow through the vertical via is allowed, and when the actuator rotates from the un-pinched position, the vertical via is pinched closed and the fluid flow is restricted.

In one embodiment, the vertical channel portions of the one or more fluidic channels in the valve body are formed in a single layer or multiple layers in the valve body.

In one embodiment, the vertical channel portion of each fluidic channel is in fluid communication with a first port and a second port.

In one embodiment, the actuator comprises one or more rolling members, and a driving member for driving the one or more rolling members to rotate independently and/or coordinately, such that when actuated, the one or more rolling members selectively address the vertical channel portions of the one or more fluidic channels so as to control rates of the fluid flow through the vertical channel portions of the one or more fluidic channels independently and/or coordinately.

In one embodiment, the one or more rolling members comprise one or more actuating discs, wherein each actuating disc is formed with a tapered or raised protrusion such that rotation of said actuating disc results in the vertical channel portion of a corresponding fluidic channel in a fully closed state, a partially closed state, or a fully open state, depending upon a position of said tapered or raised protrusion relative to the vertical channel portion.

In one embodiment, the one or more actuating discs are concentrically stacked to one another.

In one embodiment, the driving member comprises one or more nesting wrenches.

In one embodiment, the one or more rolling members comprise a plurality of rollers or a plurality of planetary gears rotatably engaged with the driving member.

In one embodiment, the driving member comprises a top plate, a bottom plate and a central hub connected to central portions of the top plate and the bottom plate, and wherein the plurality of rollers is rotatably affixed to edge portions of the top plate and the bottom plate, such that rotation of the central hub rotates causes each roller to rotate around its own axis but not translate relative to the top plate and the bottom plate.

In one embodiment, the driving member comprises a top plate, a bottom plate, a driving shaft connected to central portions of the top plate and the bottom plate, and a sun gear affixed to the driving shaft and situated between the top plate and the bottom plate, wherein each planetary gear is meshed with the sun gear and rotatably affixed between the top plate and the bottom plate, such that the rotation of the drive shaft is transferred to the sun gear, which in turn causes each planetary gear to rotate around its own axis but not translate relative to the top plate and the bottom plate.

In one embodiment, the actuator comprises: one or more actuating pins accommodated in a pin housing and corresponding to the vertical channel portions of the one or more fluidic channels, wherein each actuating pin is movable from a first position in the pin housing to a second position out of the pin housing when pushed, otherwise, said actuating pin returns from the second position to the first position, wherein when said actuating pin is in the first position, it un-pinches a corresponding vertical channel portion so that the fluid flow therethrough is not restricted, and when said actuating pin is in the second position, it pinches the corresponding vertical channel portion so that the fluid flow therethrough is restricted; a pin actuator cam having at least one lobe; and a driving shaft engaged with the pin actuator cam to operably rotate the pin actuator cam, such that when the lobe is aligned with an actuating pin, the lobe pushes said actuating pin to move from the first position to the second positon, thereby restricting the fluid flow through said corresponding vertical channel portion, when the lobe is misaligned with said actuating pin, said actuating pin returns from the second position to the first position, thereby, unrestricting the fluid flow through said corresponding vertical channel portion.

In one embodiment, each actuating pin is accommodated in the pin housing with an elastic member including a spring.

In one embodiment, the return force on the actuator pin is provided by the elastomeric material of the valve body, including PDMS.

In one embodiment, the VVRV is a mixing valve.

In one embodiment, the valve body is disposable, while the actuator is never in contact with fluid and is reusable.

In another aspect, the invention relates to a gravity perfused bioreactor, comprising: a plurality of chambers vertically stacked to one another on a substrate; and a plurality of reservoirs disposed over the plurality of chambers, wherein each chamber has an input port and an output port fluidically coupled to two corresponding reservoirs of the plurality of reservoirs, respectively; and wherein the plurality of reservoirs and the plurality of chambers are configured such that fluid flows between the plurality of reservoirs and the plurality of chambers are gravity-fed.

In one embodiment, the gravity perfused bioreactor is devoid of a valve.

In one embodiment, the gravity perfused bioreactor further comprises a plurality of permeable membranes, wherein each permeable membrane is disposed between two vertically adjacent chambers.

In one embodiment, the plurality of chambers comprises an endothelial chamber and an epithelial chamber.

In yet another aspect, the invention relates to a gravity perfused bioreactor, comprising: a ribbon fluidics comprising supply channels, collection channels, and a fluid manifold fluidically coupled between the supply channels the collection channels; a bioreactor disk attached to the ribbon fluidics, comprising a bioreactor fluidically coupled to the fluid manifold of the ribbon fluidics; a plurality of supply reservoirs fluidically coupled to the supply channels of the ribbon fluidics; and a plurality of collection reservoirs fluidically coupled to the collection channels of the ribbon fluidics, wherein the plurality of supply and collection reservoirs and the bioreactor are configured such that fluid flows between the plurality of supply and collection reservoirs and the bioreactor are gravity-fed.

In one embodiment, the gravity perfused bioreactor is devoid of a valve.

In one embodiment, each reservoir comprises an intubation recess.

In one embodiment, the ribbon fluidics comprises an inverted, twisted ribbon fluidics.

In one embodiment, the ribbon fluidics is adapted for invertible loading.

In one embodiment, the supply reservoirs, the bioreactor, and the collection reservoirs are terraced for passive pumping.

In one embodiment, the gravity perfused bioreactor further comprises an openable puck housing and an openable puck compression ring coupled to the bioreactor.

In one embodiment, the bioreactor is mounted on a microscope slide.

In one embodiment, the supply reservoirs are situated at a higher elevation than the collection reservoirs.

In one embodiment, the bioreactor comprises a plurality of bioreactor layers vertically stacked to one another, which in turn is detachably attached to the fluid manifold.

In one embodiment, the gravity perfused bioreactor further comprises a plurality of vertical-via rotary valves (VVRVs), wherein each VVRV is fluidically coupled between one of the supply reservoirs and the collection reservoirs and the bioreactor.

In one embodiment, the gravity perfused bioreactor further comprises at least one pump for inducing the fluid flows between the plurality of reservoirs and the plurality of bioreactor chambers.

In one embodiment, the gravity perfused bioreactor further comprises external pressure or suction sources including mechanical pumps or external gravity-feed/collection reservoirs.

In one embodiment, the flow rates and pressures are adjustable on-the-fly by changing the relative pressure differential between the supply and collection reservoirs by adjusting the relative elevation of supply fluid of the supply reservoirs to collection fluid of the collection reservoirs.

In one embodiment, the flow rates and pressures are adjustable by changing perfusate volume in the reservoirs or elevating the supply reservoirs relative to the collection reservoirs.

In a further aspect, the invention relates to a gravity perfused bioreactor, comprising: a plurality of bioreactor chambers stacked to one another, wherein each chamber has an input port and an output port; a plurality of reservoirs disposed over the plurality of bioreactor chambers; and a plurality of vertical-via rotary valves (VVRVs), wherein each VVRV is disclosed above and fluidically coupled between a corresponding reservoirs of the plurality of reservoirs and one of the input port and the output port of a corresponding chamber of the plurality of bioreactor chambers.

In one embodiment, the gravity perfused bioreactor further comprises a plurality of permeable membranes, wherein each permeable membrane is disposed between two adjacent bioreactor chambers.

In one embodiment, fluid flows between the plurality of reservoirs and the plurality of bioreactor chambers are gravity-fed, and/or induced by mechanical pumping.

In one embodiment, the gravity perfused bioreactor further comprises at least one pump for inducing the fluid flows between the plurality of reservoirs and the plurality of bioreactor chambers.

In one embodiment, the gravity perfused bioreactor further comprises external pressure or suction sources including mechanical pumps or external gravity-feed/collection reservoirs.

In one aspect, the invention relates to a gravity perfused bioreactor system, comprising: at least one bioreactor chamber; and a plurality of reservoirs, wherein the plurality of reservoirs comprises an autofilling supply reservoir, a bioreactor supply reservoir, and a bioreactor collection reservoir; wherein the at least one bioreactor chamber is fluidically coupled between the bioreactor supply reservoir and the bioreactor collection reservoir; and wherein the autofilling supply reservoir is fluidically coupled to the bioreactor supply reservoir by a reservoir delivery tube and a level-sensing air vent and configured with adjustable level control for a constant pressure head, such that media in the autofilling supply reservoir is released only when a fluid level in the bioreactor supply reservoir drops below the lower end of the level-sensing air vent, at which point air enters the autofilling supply reservoir, increases a volume of air trapped in the autofilling supply reservoir, and allows fluid to move from the autofilling supply reservoir to the bioreactor supply reservoir through the reservoir delivery tube.

In one embodiment, the media from the bioreactor supply reservoir then flows through the at least one bioreactor chamber into the bioreactor collection reservoir, thereby increasing the level of media in the bioreactor collection reservoir.

In one embodiment, the gravity perfused bioreactor system further comprises a vertical adjustment means coupled to the autofilling supply reservoir for setting the fluid height in the bioreactor supply reservoir.

In one embodiment, the autofilling supply reservoir is situated over the bioreactor supply reservoir, or located remote from the bioreactor supply reservoir.

In one embodiment, the gravity perfused bioreactor system further comprises a wick fluidically coupled to the bioreactor collection reservoir for adjusting the fluid level in the bioreactor collection reservoir.

In one embodiment, the wick comprises a collection bag, a collection tube having one end placed in the bioreactor collection reservoir and the other end placed in the collection bag, and a collection wick received in the collection tube.

In one embodiment, the gravity perfused bioreactor system further comprises a siphon tube inserted in a side punched hole of the bioreactor collection reservoir for adjusting the fluid level in the bioreactor collection reservoir.

In one embodiment, the autofilling supply reservoir is a large-volume airtight reservoir that maintains a constant supply reservoir height therein, and the wick or tube collection system ensures that the collection fluid in the bioreactor collection reservoir has a near-constant height so that media flows from the airtight autofilling reservoir, through the bioreactor supply reservoir, and perfuses the bioreactor at a constant flow rate.

In another aspect, the invention relates to a gravity perfused bioreactor system, comprising: a first reservoir and a second reservoir; at least one bioreactor chamber having at least one first channel fluidically coupled to the first reservoir, and at least one second channel fluidically coupled to the second reservoir; and a valve comprising: at least one first valve pin situated on the at least one first channel between the first reservoirs and the at least one bioreactor chamber, such that when the at least one first valve pin is pushed, fluid flow through the at least one first channel is occluded, and when the at least one first valve pin is released, the fluid flow through the at least one first channel is allowed; at least one second valve pin situated on the at least one second channel between the at least one bioreactor chamber and the second reservoir, such that when the at least one second valve pin is pushed, the fluid flow through the at least one second channel is occluded, and when the at least one second valve pin is released, the fluid flow through the at least one second channel is allowed; and an actuator having an actuator head for pushing or releasing the at least one first valve pin and the at least one second valve pin, such that when the gravity perfused bioreactor system is tilted in one direction, the actuator head pushes the at least one first valve pin and releases the at least one second valve pin so as to occlude fluid flow through the at least one first channel and allow fluid flow through the at least one second channel, and when the gravity perfused bioreactor system is tilted in an opposite direction, the actuator head releases the at least one first valve pin and pushes the at least one second valve pin so as to allow fluid flow through the at least one first channel and occlude fluid flow through the at least one second channel.

In one embodiment, the actuator is a rocker

In one embodiment, a rigid support is coupled to the rocker at an end opposite the actuator head for alignment with the center of the rocker.

In one embodiment, the rocker is an inverted pendulum that is bistable and movable from one side to the other as the system is tilted back and forth.

In yet another aspect, the invention relates to a gravity perfusion bioreactor system, comprising: an enclosure for cell cultures, comprising a slidable drawer/carrier; a plurality of fully enclosed, tilted reservoirs disposed in the enclosure for long-duration gravity perfusion of multiple independent bioreactors; and a modular array of gravity fed multiple independent bioreactors supported by a slidable drawer/carrier, using gravity and vertical separation to maintain consistent flow to the multiple independent bioreactors without requiring electrical power.

In one embodiment, each module includes two NVUs resting in a containment tray.

In one embodiment, fluid flows from supply containers through tubing to inputs of the multiple independent bioreactors and from outputs of the multiple independent bioreactors through tubing to waste containers.

In one embodiment, the enclosure has an HEPA filter window for atmosphere exchange, or is sealed to prevent contamination.

In one embodiment, the reservoirs comprise bag perfusion and collection reservoirs, wherein the system utilizes gravity to provide near-constant perfusion rates over for very long experiments using gas-permeable bags.

In one embodiment, the reservoirs are angled so as to maximize useful fluid volume while minimizing pressure differential as the fluid drains through the experiment.

In one embodiment, the supply and collection vials are arrayed beneath the NVU pucks.

In one embodiment, the supply and collection vials are vented and tilted.

In one embodiment, the multiple independent bioreactors are a high-density array of bioreactors with a motorized multi-channel pump running off a sealed gel lead-acid storage battery.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A-1B show conventional gravity-perfused bioreactors. FIG. 1A: Four gravity perfused bioreactors fed by a common reservoir with an output manifold for collecting waste media and restricting flow rate without the explicit use of a valve. FIG. 1B: A horizontal control valve embedded within a microfluidic device that would be connected to a biorector.

FIG. 2A-2E show conventional on-chip valves. FIGS. 2A-2C: A tape underlayment rotary node (TURN) valve. FIG. 2D: A rotary microfluidic valve assembly with a rigid rotor that contains channels. Seals on the edge or top prevent leaks. FIG. 2E: A microfluidic sliding valve assembly.

FIGS. 3A-3E show a typical two-chamber, airway barrier bioreactor. FIGS. 3A-3B: two cross-sectional views. FIGS. 3C-3E: three perspective views, including two containers for secondary containment.

FIGS. 4A-4B show schematically an airway-on-chip device according to embodiments of the invention. Endothelial and epithelial networks are identical, such that both physical layers can be produced with a single mask. FIG. 4A: Endothelial (solid) and epithelial (dashed) layers, superimposed. FIG. 4B: Mask layout for multiple devices with reservoir footprints and vertical-via rotary valves.

FIG. 5 shows schematically a common mask for the perfusion network for both chambers of a two-chamber bioreactor according to embodiments of the invention.

FIGS. 6A-6H show a bioreactor and vertical-via rotary valves according to embodiments of the invention. FIGS. 6A-6B: Transparently perspective views of the bioreactor with a vertical-via rotary valve. FIG. 6C: The actuator for a vertical-via rotary valve. FIG. 6D-6F: Three operational top views of the actuator of the vertical-via rotary valve in the valve body. FIGS. 6G-6H: Two operational section views of the vertical-via rotary valve.

FIGS. 7A-7C show three different examples of lobe shapes for a vertical-via rotary valve according to embodiments of the invention.

FIGS. 8A-8C show three different partial views of an actuator for a vertical-via rotary metering valve and its relationship with vias and microfluidic channels according to embodiments of the invention.

FIGS. 9A-9E show five different views of a valve body of a vertical-via rotary valve according to embodiments of the invention.

FIGS. 10A-10B show a vertical-via rotary valve according to embodiments of the invention. FIG. 10A: Four views of an actuator for a vertical-via rotary valve. FIG. 10B: Operation of the vertical-via rotary valve.

FIGS. 11A-11D show via, actuator and channel layer embodiments with two different actuators according to embodiments of the invention.

FIGS. 12A-12C show different views of a mixing vertical-via rotary valve according to embodiments of the invention.

FIGS. 13A-13G shows independent coaxial actuators with concentric alignment grooves according to embodiments of the invention.

FIGS. 14A-14D show different views of a nesting wrench for independent control of each actuator according to embodiments of the invention. A coupled wrench could provide coordinated actuation.

FIGS. 15A-15C show the use of an unvalved second via to return the flow back down to the lower fluidics layer to provide tubing support according to embodiments of the invention.

FIGS. 16A-16B show different views of a closed and throttled stacked, multiport vertical-via rotary valve according to embodiments of the invention.

FIGS. 17A-17B show different views of a fluidic layout for a four-channel rotary vertical-via rotary valve according to embodiments of the invention.

FIGS. 18A-18D show different views of a roller actuator according to embodiments of the invention.

FIGS. 19A-19E show different views of a planetary gear actuator according to embodiments of the invention.

FIGS. 20A-20D show different views of a gravity-perfused bioreactors without valves according to embodiments of the invention.

FIGS. 21A-21E show different views of a gravity perfused bioreactor with on-board vertical-via rotary valves according to embodiments of the invention.

FIGS. 22A-22C show different views of an invertible, two-sided bioreactor with on-board vertical-via rotary valves according to embodiments of the invention.

FIGS. 23A-23C show different embodiments using an eccentric or lobed valve actuator with spring-return pins according to embodiments of the invention.

FIG. 24 illustrates the loading sequence for a two-layer airway barrier chip according to embodiments of the invention.

FIGS. 25A-25I show different gravity perfusion systems with ribbon fluidics for invertible loading according to embodiments of the invention.

FIGS. 26A-26F show different views of a gravity perfused manifold with a detachable bioreactor according to embodiments of the invention.

FIGS. 27A-27D show different views of ribbon fluidics with either transverse or vertical valves according to embodiments of the invention.

FIGS. 28A-28D show different views of self-filling reservoirs with adjustable level control and wicking drain for constant pressure head according to embodiments of the invention.

FIGS. 29A-29F show different views of tilt-perfused, unidirectional, valved, gravity perfusion system according to embodiments of the invention.

FIGS. 30A-30F show different views of tilted reservoirs for long-duration gravity perfusion within a containment drawer according to embodiments of the invention.

FIGS. 31A-31C show different views of containment drawer enclosing a high-density array with motorized multi-channel pump running off a sealed gel lead-acid storage battery according to embodiments of the invention.

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially” or “approximately” shall generally mean within 20 percent, preferably within 10 percent, and more preferably within 5 percent of a given value or range. Numerical quantities given herein are approximate, meaning that the terms “around,” “about,” “substantially” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. As used herein, the term “and/or” includes any and all combinations of one or more of the associated listed items. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Often gravity-induced flow through a microfluidic network or a bioreactor is a practical and very inexpensive alternative to using complicated and bulky pumps. For applications that do not require fast flows or repeated adjustments of the flow rate, or where the use of external pumping devices is cost- or space-prohibitive, gravity-induced flow offers a simple solution. The chip can contain the supply and collection reservoirs and be tilted to provide the height difference between the two; without tilting, open, vertical pipette tips can be inserted into the fluidic channels, or, also without tilting, cylindrical glass reservoirs can be bonded to the chip to deliver fluid and store waste, with the flow rate depending upon the height difference between the supply and waste reservoirs.

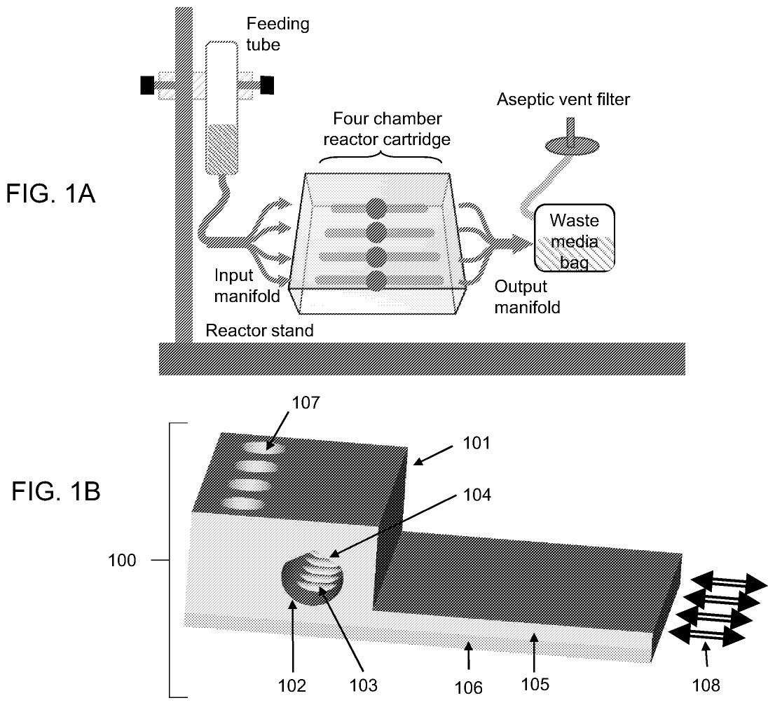

The primary advantage of gravity perfusion of microfluidic bioreactors is that it does not require electrical power or mechanical pumps or external tubing. FIGS. 1A-1B shows conventional gravity perfused bioreactor systems with on-chip valves. FIG. 1A shows four gravity perfused bioreactors are fed by a common reservoir with an output manifold for restricting flow rate and collecting waste media into a common bag. FIG. 1B shows a horizontal control valve embedded within a microfluidic device, for example the end of a ribbon microfluidic connected to a bioreactor. The end of the microfluidic interconnect system 100 can have an integrally cast valve body 101 with a molded cavity 102 in which the rotary cylindrical valve actuator operates. Within the cavity, there are raised regions 103 beneath which the fluidic conduits are located. These conduits connect the ports 107 to the internal conduits that carry fluids 108 to the connected microfluidic bioreactor, perfusion controller, or other instrument. Planar surfaces 104 at the top of the cavity maintain the alignment of the cylindrical valve actuator as it rotates and delivers compressive forces to the conduits underneath 103. The conduits are formed in either the lower surface 105 of the valve body or the upper surface of the membrane 106 that seals the conduits. The key feature of this approach is that the valve has only two parts—the valve body that is integral to the microfluidic interconnect, and the single-piece valve body. Both can be cast or injection molded, the former from a flexible elastomer and the latter from a rigid material. This greatly simplifies the fabrication and reduces the cost of the cut-off valve that would be part of the bioreactor fluidics discussed above. A cylindrical actuator with detents (not shown) is inserted into the cavity 102 such that rotation of the actuator can compress the raised regions to control the fluidic channels beneath ridges 103.

Neither system addresses the need for a compact, readily contained bioreactor perfusion system suitable for operation in a BSL-3 or BSL 4 environment as is required for the study of infectious agents and their interactions with cells cultured in microfluidic microbioreactors.

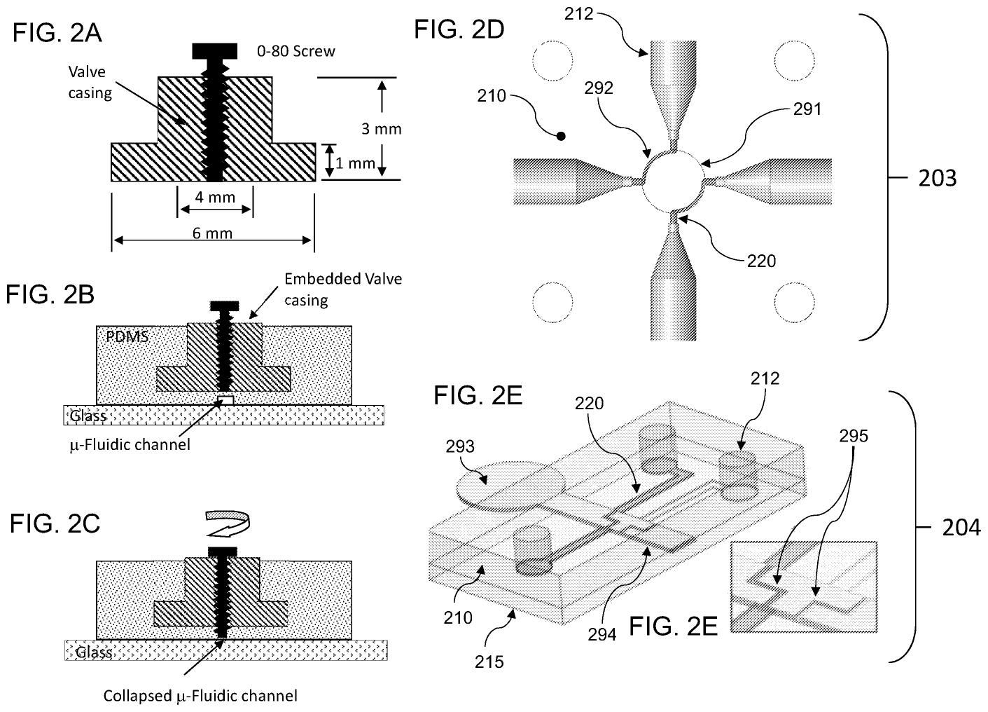

FIGS. 2A-2E provides three examples of other on-chip valves. FIGS. 2A-2C provide a tape underlayment rotary node (TURN) valve, which has an 0-80 machine screw threaded through a valve casing and embedded within the bulk elastomer of a PDMS microfluidic chip that is bonded to a glass microscope slide. The microfluidic channel between the screw and the glass is open in FIG. 2B, but as the screw is turned and pushed down on the PDMS, the end of the screw compresses the PDMS and collapses and seals the channel (FIG. 2C). Beyond the difficulty of fabricating the TURN valve, it has the major disadvantage that overtightening can cause delamination of the PDMS valve body from the glass substrate, with the distortion forces required to close the valve having to be applied in the immediate vicinity of a bond between two parts of the valve body. Earlier, simpler versions of this approach, termed a torque activated or Twist valve, have been described in the literature and suffer from these and other limitations.

FIG. 2D shows a rotary microfluidic valve assembly 203 in which valve body 210 has four ports 212 in the valve body for external tubing. Rotating valve actuator plug 291 with circumferential channel 292 enables connection between fluidic channels in the valve body 220 and the two external tubing ports 212. Rotating the valve actuator plug by 90 degrees sequentially connects different pairs of tubing ports. The greatest disadvantage of this type of design is that the interface between the actuator plug 291 and the valve body 210 can in fact be contaminated by the substances carried through any of the four ports. In addition, seals on the top and bottom are required to prevent leaks.

FIG. 2E shows a microfluidic sliding valve assembly 204 where a sliding valve actuator 293 operates in a groove 294 in valve body 210 above glass or plastic substrate 215. Channels 295 in the sliding actuator connect channels 220 with tubing ports 212 as specified by the configuration of the channels 295. Seals on the edge or top prevent leaks. In addition to the potential risks of leaks and cross-contamination, this valve requires side access to the chip, which may complicate actuation and limit the possible number of different valve configurations that can be readily implemented in a single chip. Neither of the valves in FIG. 2D or 2E are suitable for metering a flow.

In the current COVID-19 pandemic arising from infections by the SARS-CoV-2 virus, in vitro models of the blood-brain, airway-lung, and gut barriers are of particular interest, but these measurements must be made in a Biosafety Level 3 (BSL-3) containment environment, which places severe technical and economic and space constraints on any instrumentation that is needed for the studies. For each of these physiological barriers in vivo, the structural, and biological integrity of the barrier membrane is critical to prevent infectious pathogens from crossing from an external environment to the internal, cellular environment where the infectious pathogens can multiply and spread. In a typical in vitro microfluidic model of one of these barriers, a semipermeable membrane is encapsulated between two bioreactor chambers, with one cell type on one side and a different cell type on the other.

In the case of the neurovascular unit (NVU) and the blood-brain barrier (BBB) that it protects, the barrier is formed by human brain microvascular endothelial cells on one side of a thin, porous membrane, and astrocytes and pericytes on the other. It is important that the vascular media have a high linear velocity to ensure proper biological polarization of the endothelial cells to create a tight BBB. In an in vitro airway-on-a-chip or an alveolar chip model, one side of the barrier has a confluent layer of the appropriate pulmonary epithelial cells, while the other side of the barrier has a confluent layer of pulmonary microvascular cells. In these lung-chip models, once the epithelial endothelial cell cultures are established, the perfusion liquid is removed from the epithelial side to create an air-liquid interface that can be used for aerosol infection of the chip with the SARS-CoV-2 or another virus. While in vitro models of the lining of the small intestine require a liquid-liquid interface across the endothelial cells, in vitro models of the colon may, in certain circumstances, require an air-liquid interface between the luminal and stromal sides of the interface. Other barrier chips, such as ones recapitulating the blood-testis barrier or the blood-skin barrier, would follow similar design principles, and a model of the fetal-maternal interface might require three barriers and four chambers. The embodiments presented in this disclosure are not meant to be limited to a particular organ model, interface nature, or number of chambers and barriers.

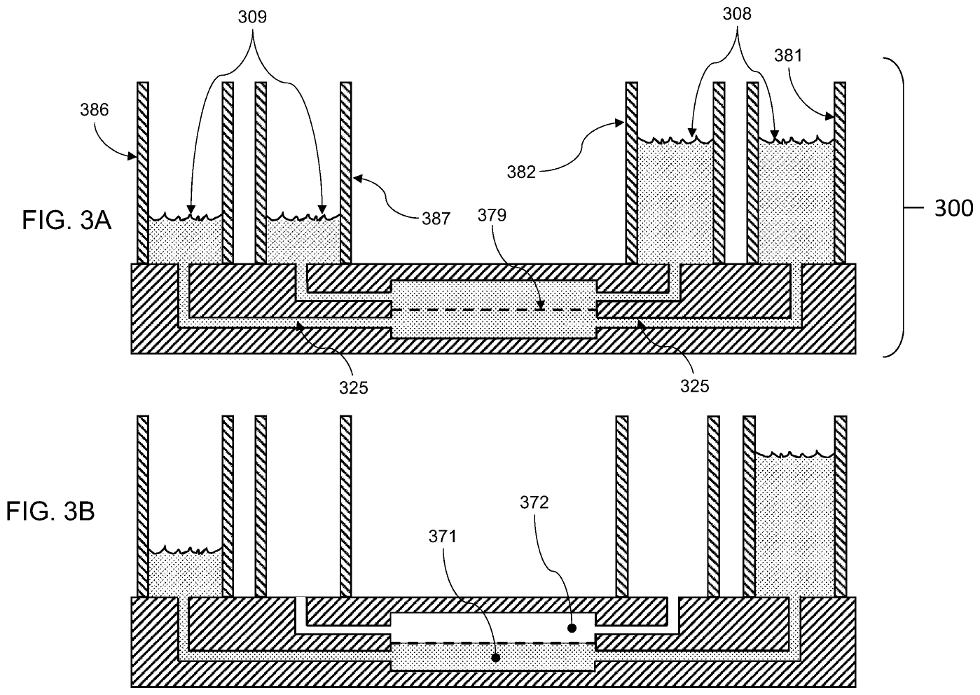

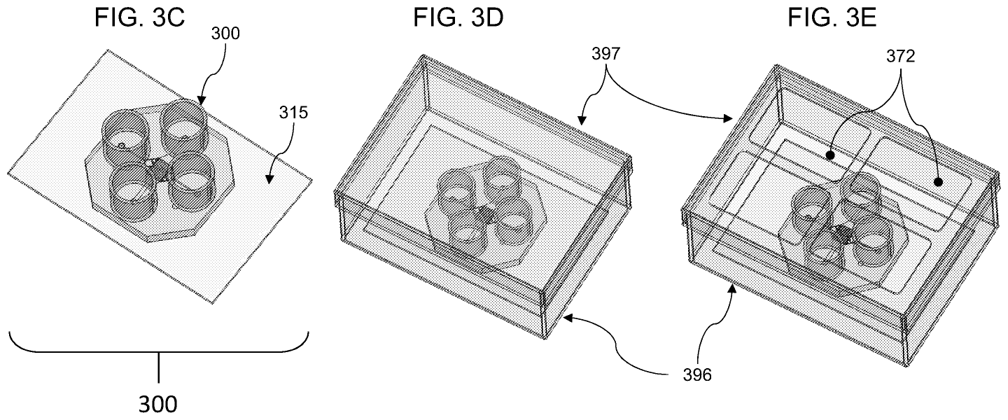

FIG. 3A shows a typical two-chamber, airway barrier bioreactor 300 with gravity perfusion. The supply media 308 for each chamber 371 and 372 is stored in the two supply reservoirs 381 (vascular) and 382 (airway), and the media that passes through the bioreactor chambers is collected in matched reservoirs 386 and 387. The flow rate past the barrier membrane 379 is determined by the difference in height of fluid in the supply (381 and 382) and collection (386 and 387) reservoirs and the hydraulic resistance of the inlet 325 and output 326 channels within the chip. As soon as the pulmonary epithelial cells are established on the membrane surface exposed to bioreactor chamber 372, the fluid is removed from that chamber to create an air-liquid interface, as shown in FIG. 3B. The endothelial cells lining vascular bioreactor chamber 371 remain perfused in FIG. 3B after the creation of the air-liquid interface, providing nutrition and moisture to the epithelial cells on the upper surface of the membrane.

The strict protocols associated with studying infectious microorganisms in a BSL-3 facility require that all fluid-containing devices have secondary confinement. FIG. 3C shows a two-chamber barrier bioreactor 300 bonded to microscope slide substrate 315. The tops of the reservoirs are open, as is required for gravity flow and adding and removing media. This device cannot be used as shown in BSL-3. The secondary containment box 396 and containment box lid 397 can enclose one or more bioreactors 310 in accordance with BSL-3 protocols. The lid in FIG. 3E shows a hydrophobic containment gas-exchange membrane 398 thermally bonded to provide gas exchange while still providing liquid and particle containment.

We have successfully used gravity perfusion in the past for long-term cell culture in a thick tissue bioreactor (TTB). A simple way to control the flow rate in the gravity driven system for a given pressure differential without tilting is to have control over fluidic resistance of the network. This approach was implemented in our TTB by introducing a constricting output manifold. By selecting a manifold with appropriate internal channel dimensions, one can produce a sufficient resistance to flow that, in combination with the fixed height of the supply reservoir, could be used to passively limit the flow through the bioreactor. Such an approach works well for a given fluidic configuration but lacks flexibility and does not guarantee uniform flows from the manifold ports. An adjustable valve that can change fluidic resistance of the flow path depending on experimental requirements would be a significant improvement and a more universal solution to the passive flow regulation problem.

We envision that such a valve coupled with a gravity (or pressure) induced flow bioreactor would be opened, adjusted, or closed only once or twice during the length of the experiment. As such this valve should be simple and easily integrated with microfluidics, have minimal footprint, and be compatible with passive/stand-alone operation without external power sources and capable of reversibly opening and closing the channels. Operational modes would include 1) completely open for device fluid and cell loading or flushing; 2) set to particular predetermined position (value) based on calibration curves and experimental parameters; and 3) closed to stop the flow, for example at the end of an experiment.

One of the objectives of this invention is to disclose a new class of gravity or pressure-perfused bioreactors that are designed specifically for use in a BSL-3 or BSL-4 environment, but that have a large number of other possible applications in drug discovery, toxicology, biology, and medicine, to meet the above-noted needs.

The rate of fluid flow in gravity perfused devices is typically controlled by adjusting the difference in the height of the fluid in the input and output reservoirs. If the vertical height of the device is limited, for example by the need for a secondary containment enclosure, then the rate of flow control can be adjusted only by changing the channel width or height within the device. If high flow rates are required, the reservoirs need to have a large volume to provide perfusion fluid for extended intervals, for example one day. If large changes in flow rate are to be avoided, the reservoir should have a large horizontal cross-sectional area so that the vertical change in fluid heights is small. While not typically used in gravity perfusion, there are a large number of different microfluidic valves that operate with either mechanical or pneumatic force to compress a channel to control flow under pumped or pressure-driven flows. In many of these valves, the applied force is perpendicular to the plane that contains the bond between an elastomeric body or membrane and a rigid substrate or channel structure, thereby presenting a force that could weaken or fracture a bond required to seal the microfluidic device against leaks. The rotary via valve described in this application is unique in that it applies a horizontal, radial force against a vertical via, so that the primary compressive forces are parallel to and removed from the bonding plane, thereby producing a valve that is less likely to rupture from repeated use or overpressurization. The advantage of this valve, when coupled with integrated supply and waste reservoirs, is that the flows can be adjusted or transiently turned off as needed for a particular MPS protocol without the need to construct a microfluidic chip that is tailored for a particular flow rate.

In one aspect, the invention relates to a vertical-via rotary valve (VVRV) comprising: a valve body having a housing; one or more fluidic channels, each fluidic channel having a vertical channel portion defined in the valve body and being adjacent to the housing, wherein a fluid flow through the vertical channel portion is controllable by deforming a sidewall of the vertical channel portion; and an actuator received in the housing and rotatably engaged with the one or more fluidic channels to operably control the fluid flow through the vertical channel portion of each fluid channel.

In certain embodiments, the valve body is formed of an elastomeric material including polydimethylsiloxane (PDMS).

In certain embodiments, the vertical channel portion of each fluidic channel is a vertical via.

In certain embodiments, the actuator features a vertical trough aligned with the vertical via, such that when the actuator un-pinches the vertical via and the fluid flow through the vertical via is allowed, and when the actuator rotates from the un-pinched position, the vertical via is pinched closed and the fluid flow is restricted.

In certain embodiments, the vertical channel portions of the one or more fluidic channels in the valve body are formed in a single layer or multiple layers in the valve body.

In certain embodiments, each channel may include horizontal portions extending from and/or fluidic coupled to the vertical channel. The vertical channel and horizontal portions of the one or more fluidic channels in the valve body are formed in a single layer or multiple layers in the valve body.

In certain embodiments, the vertical channel parts are only in the central portion. The channels that lead into or out of the vertical via could be in the other layers. That said, one might make a vertical via valve that in fact requires two body layers.

In certain embodiments, both the vertical and horizontal channels are in the central portion.

In certain embodiments, the vertical channel portion of each fluidic channel is in fluid communication with a first port and a second port.

In certain embodiments, the actuator comprises one or more rolling members, and a driving member for driving the one or more rolling members to rotate independently and/or coordinately, such that when actuated, the one or more rolling members selectively address the vertical channel portions of the one or more fluidic channels so as to control rates of the fluid flow through the vertical channel portions of the one or more fluidic channels independently and/or coordinately.

In certain embodiments, the one or more rolling members comprise one or more actuating discs, wherein each actuating disc is formed with a tapered or raised protrusion such that rotation of said actuating disc results in the vertical channel portion of a corresponding fluidic channel in a fully closed state, a partially closed state, or a fully open state, depending upon a position of said tapered or raised protrusion relative to the vertical channel portion.

In certain embodiments, the one or more actuating discs are concentrically stacked to one another.

In certain embodiments, the driving member comprises one or more nesting wrenches.

In certain embodiments, the one or more rolling members comprise a plurality of rollers or a plurality of planetary gears rotatably engaged with the driving member.

In certain embodiments, the driving member comprises a top plate, a bottom plate and a central hub connected to central portions of the top plate and the bottom plate, and wherein the plurality of rollers is rotatably affixed to edge portions of the top plate and the bottom plate, such that rotation of the central hub rotates causes each roller to rotate around its own axis but not translate relative to the top plate and the bottom plate.

In certain embodiments, the driving member comprises a top plate, a bottom plate, a driving shaft connected to central portions of the top plate and the bottom plate, and a sun gear affixed to the driving shaft and situated between the top plate and the bottom plate, wherein each planetary gear is meshed with the sun gear and rotatably affixed between the top plate and the bottom plate, such that the rotation of the drive shaft is transferred to the sun gear, which in turn causes each planetary gear to rotate around its own axis but not translate relative to the top plate and the bottom plate.

In certain embodiments, the actuator comprises: one or more actuating pins accommodated in a pin housing and corresponding to the vertical channel portions of the one or more fluidic channels, wherein each actuating pin is movable from a first position in the pin housing to a second position out of the pin housing when pushed, otherwise, said actuating pin returns from the second position to the first position, wherein when said actuating pin is in the first position, it un-pinches a corresponding vertical channel portion so that the fluid flow therethrough is not restricted, and when said actuating pin is in the second position, it pinches the corresponding vertical channel portion so that the fluid flow therethrough is restricted; a pin actuator cam having at least one lobe; and a driving shaft engaged with the pin actuator cam to operably rotate the pin actuator cam, such that when the lobe is aligned with an actuating pin, the lobe pushes said actuating pin to move from the first position to the second positon, thereby restricting the fluid flow through said corresponding vertical channel portion, when the lobe is misaligned with said actuating pin, said actuating pin returns from the second position to the first position, thereby, unrestricting the fluid flow through said corresponding vertical channel portion.

In certain embodiments, each actuating pin is accommodated in the pin housing with an elastic member including a spring.

In certain embodiments, the return force on the actuator pin is provided by the elastomeric material of the valve body, including PDMS.

In certain embodiments, the VVRV is a mixing valve.

In certain embodiments, the valve body is disposable, while the actuator is never in contact with fluid and is reusable.

In another aspect, the invention relates to a gravity perfused bioreactor, comprising: a plurality of chambers vertically stacked to one another on a substrate; and a plurality of reservoirs disposed over the plurality of chambers, wherein each chamber has an input port and an output port fluidically coupled to two corresponding reservoirs of the plurality of reservoirs, respectively; and wherein the plurality of reservoirs and the plurality of chambers are configured such that fluid flows between the plurality of reservoirs and the plurality of chambers are gravity-fed.

In certain embodiments, the gravity perfused bioreactor is devoid of a valve.

In certain embodiments, the gravity perfused bioreactor further comprises a plurality of permeable membranes, wherein each permeable membrane is disposed between two vertically adjacent chambers.

In certain embodiments, the plurality of chambers comprises an endothelial chamber and an epithelial chamber.

In yet another aspect of the invention, the gravity perfused bioreactor comprises: a ribbon fluidics comprising supply channels, collection channels, and a fluid manifold fluidically coupled between the supply channels the collection channels; a bioreactor disk attached to the ribbon fluidics, comprising a bioreactor fluidically coupled to the fluid manifold of the ribbon fluidics; a plurality of supply reservoirs fluidically coupled to the supply channels of the ribbon fluidics; and a plurality of collection reservoirs fluidically coupled to the collection channels of the ribbon fluidics, wherein the plurality of supply and collection reservoirs and the bioreactor are configured such that fluid flows between the plurality of supply and collection reservoirs and the bioreactor are gravity-fed.

In certain embodiments, the gravity perfused bioreactor is devoid of a valve.

In certain embodiments, each reservoir comprises an intubation recess.

In certain embodiments, the ribbon fluidics comprises an inverted, twisted ribbon fluidics.

In certain embodiments, the ribbon fluidics is adapted for invertible loading.

In certain embodiments, the supply reservoirs, the bioreactor, and the collection reservoirs are terraced for passive pumping.

In certain embodiments, the gravity perfused bioreactor further comprises an openable puck housing and an openable puck compression ring coupled to the bioreactor.

In certain embodiments, the bioreactor is mounted on a microscope slide.

In certain embodiments, the supply reservoirs are situated at a higher elevation than the collection reservoirs.

In certain embodiments, the bioreactor comprises a plurality of bioreactor layers vertically stacked to one another, which in turn is detachably attached to the fluid manifold.

In certain embodiments, the gravity perfused bioreactor further comprises a plurality of VVRVs. Each VVRV is fluidically coupled between one of the supply reservoirs and the collection reservoirs and the bioreactor.

In certain embodiments, the gravity perfused bioreactor further comprises at least one pump for inducing the fluid flows between the plurality of reservoirs and the plurality of bioreactor chambers.

In certain embodiments, the gravity perfused bioreactor further comprises external pressure or suction sources including mechanical pumps or external gravity-feed/collection reservoirs.

In certain embodiments, the flow rates and pressures are adjustable on-the-fly by changing the relative pressure differential between the supply and collection reservoirs by adjusting the relative elevation of supply fluid of the supply reservoirs to collection fluid of the collection reservoirs.

In certain embodiments, the flow rates and pressures are adjustable by changing perfusate volume in the reservoirs or elevating the supply reservoirs relative to the collection reservoirs.

In a further aspect of the invention, the gravity perfused bioreactor comprises: a plurality of bioreactor chambers stacked to one another, wherein each chamber has an input port and an output port; a plurality of reservoirs disposed over the plurality of bioreactor chambers; and a plurality of VVRVs. Each VVRV is disclosed above and fluidically coupled between a corresponding reservoirs of the plurality of reservoirs and one of the input port and the output port of a corresponding chamber of the plurality of bioreactor chambers.

In certain embodiments, the gravity perfused bioreactor further comprises a plurality of permeable membranes, wherein each permeable membrane is disposed between two adjacent bioreactor chambers.

In certain embodiments, fluid flows between the plurality of reservoirs and the plurality of bioreactor chambers are gravity-fed, and/or induced by mechanical pumping.

In certain embodiments, the gravity perfused bioreactor further comprises at least one pump for inducing the fluid flows between the plurality of reservoirs and the plurality of bioreactor chambers.

In certain embodiments, the gravity perfused bioreactor further comprises external pressure or suction sources including mechanical pumps or external gravity-feed/collection reservoirs.

In one aspect of the invention, the gravity perfused bioreactor system comprises: at least one bioreactor chamber; and a plurality of reservoirs, wherein the plurality of reservoirs comprises an autofilling supply reservoir, a bioreactor supply reservoir, and a bioreactor collection reservoir; wherein the at least one bioreactor chamber is fluidically coupled between the bioreactor supply reservoir and the bioreactor collection reservoir; and wherein the autofilling supply reservoir is fluidically coupled to the bioreactor supply reservoir by a reservoir delivery tube and a level-sensing air vent and configured with adjustable level control for a constant pressure head, such that media in the autofilling supply reservoir is released only when a fluid level in the bioreactor supply reservoir drops below the lower end of the level-sensing air vent, at which point air enters the autofilling supply reservoir, increases a volume of air trapped in the autofilling supply reservoir, and allows fluid to move from the autofilling supply reservoir to the bioreactor supply reservoir through the reservoir delivery tube.

In certain embodiments, the media from the bioreactor supply reservoir then flows through the at least one bioreactor chamber into the bioreactor collection reservoir, thereby increasing the level of media in the bioreactor collection reservoir.

In certain embodiments, the gravity perfused bioreactor system further comprises a vertical adjustment means coupled to the autofilling supply reservoir for setting the fluid height in the bioreactor supply reservoir.

In certain embodiments, the autofilling supply reservoir is situated over the bioreactor supply reservoir, or located remote from the bioreactor supply reservoir.

In certain embodiments, the gravity perfused bioreactor system further comprises a wick fluidically coupled to the bioreactor collection reservoir for adjusting the fluid level in the bioreactor collection reservoir.

In certain embodiments, the wick comprises a collection bag, a collection tube having one end placed in the bioreactor collection reservoir and the other end placed in the collection bag, and a collection wick received in the collection tube.

In certain embodiments, the gravity perfused bioreactor system further comprises a siphon tube inserted in a side punched hole of the bioreactor collection reservoir for adjusting the fluid level in the bioreactor collection reservoir.

In certain embodiments, the autofilling supply reservoir is a large-volume airtight reservoir that maintains a constant supply reservoir height therein, and the wick or tube collection system ensures that the collection fluid in the bioreactor collection reservoir has a near-constant height so that media flows from the airtight autofilling reservoir, through the bioreactor supply reservoir, and perfuses the bioreactor at a constant flow rate.

In another aspect of the invention, the gravity perfused bioreactor system comprises: a first reservoir and a second reservoir; at least one bioreactor chamber having at least one first channel fluidically coupled to the first reservoir, and at least one second channel fluidically coupled to the second reservoir; and a valve comprising: at least one first valve pin situated on the at least one first channel between the first reservoirs and the at least one bioreactor chamber, such that when the at least one first valve pin is pushed, fluid flow through the at least one first channel is occluded, and when the at least one first valve pin is released, the fluid flow through the at least one first channel is allowed; at least one second valve pin situated on the at least one second channel between the at least one bioreactor chamber and the second reservoir, such that when the at least one second valve pin is pushed, the fluid flow through the at least one second channel is occluded, and when the at least one second valve pin is released, the fluid flow through the at least one second channel is allowed; and an actuator having an actuator head for pushing or releasing the at least one first valve pin and the at least one second valve pin.

As such, when the gravity perfused bioreactor system is tilted in one direction, the actuator head pushes the at least one first valve pin and releases the at least one second valve pin so as to occlude fluid flow through the at least one first channel and allow fluid flow through the at least one second channel, and when the gravity perfused bioreactor system is tilted in an opposite direction, the actuator head releases the at least one first valve pin and pushes the at least one second valve pin so as to allow fluid flow through the at least one first channel and occlude fluid flow through the at least one second channel.

In certain embodiments, the actuator is a rocker

In certain embodiments, a rigid support is coupled to the rocker at an end opposite the actuator head for alignment with the center of the rocker.

In certain embodiments, the rocker is an inverted pendulum that is bistable and movable from one side to the other as the system is tilted back and forth.

In yet another aspect of the invention, the gravity perfused bioreactor system comprises: an enclosure for cell cultures, comprising a slidable drawer/carrier; a plurality of fully enclosed, tilted reservoirs disposed in the enclosure for long-duration gravity perfusion of multiple independent bioreactors; and a modular array of gravity fed multiple independent bioreactors supported by a slidable drawer/carrier, using gravity and vertical separation to maintain consistent flow to the multiple independent bioreactors without requiring electrical power.

In certain embodiments, each module includes two NVUs resting in a containment tray.

In certain embodiments, fluid flows from supply containers through tubing to inputs of the multiple independent bioreactors and from outputs of the multiple independent bioreactors through tubing to waste containers.

In certain embodiments, the enclosure has an HEPA filter window for atmosphere exchange, or is sealed to prevent contamination.

In certain embodiments, the reservoirs comprise bag perfusion and collection reservoirs, wherein the system utilizes gravity to provide near-constant perfusion rates over for very long experiments using gas-permeable bags.

In certain embodiments, the reservoirs are angled so as to maximize useful fluid volume while minimizing pressure differential as the fluid drains through the experiment.

In certain embodiments, the supply and collection vials are arrayed beneath the NVU pucks.

In certain embodiments, the supply and collection vials are vented and tilted.

In certain embodiments, the multiple independent bioreactors are a high-density array of bioreactors with a motorized multi-channel pump running off a sealed gel lead-acid storage battery.

In certain embodiments, a gravity perfused bioreactor can be seeded initially upright and/or inverted by a tubing connection to a larger gravity reservoir, a syringe pump, or an external peristaltic pump and then, after the tubing is removed from the bottom of the reservoirs, the device can be inverted and the now-upright reservoirs can be filled to support gravity perfusion.

In certain embodiments, a gravity perfused airway bioreactor has reservoirs on opposite sides so that the cells on the airway side can be perfused during and after seeding, and the device then inverted to operate the airway side as an air-liquid interface while the opposite, endothelial side is gravity perfused and seeded.

In certain embodiments, a vertical-via rotary valve (VVRV) allows regulation of the rate of flow from an integral supply reservoir through an MPS microbioreactor into a collection reservoir.

In certain embodiments, the valve body is designed to be disposable, single use, while the actuator, which is never in contact with fluid, can be reused.

In certain embodiments, a VVRV provides independent control of multiple fluid flows.

In certain embodiments, a VVRV provides coupled control of two or more fluid flows to comprise a mixing valve.

In certain embodiments, a VVRV utilizes rollers within the VVRV actuator to minimize friction.

In certain embodiments, a VVRV has an eccentric cam actuator.

In certain embodiments, a VVRV uses actuator pins.

In certain embodiments, a VVRV actuator is geared to provide both additional torque and finer control of flow rate.

In certain embodiments, a gravity perfused bioreactor with one, two, or more chambers, separated by semipermeable membranes, is connected to integrated perfusion reservoirs by a flexible ribbon fluidic that allows the orientation of the bioreactor to be inverted without inverting the reservoirs, and allows the relative height of the reservoirs to be adjusted to control flow rate.

In certain embodiments, a multi-chamber bioreactor can be permanently bonded or reversibly clamped to a flexible-fluidic gravity perfused manifold, so that the design and fabrication of the bioreactor and the fluid manifold are decoupled.

In certain embodiments, in a gravity perfused bioreactor, the level of fluid in the supply reservoir is maintained by a second reservoir from which fluid flows based upon the height of fluid in the supply reservoir.

In certain embodiments, in a gravity perfused bioreactor, the level of fluid in the collection reservoir is determined by the height of a wick in the collection reservoir, with the wick delivering the collected fluid into a lower secondary collection reservoir or bag.

In certain embodiments, a gravity perfused tilting bioreactor for which the direction of flow between two reservoirs is controlled by a valve whose operation is controlled by the tilting operation to provide unidirectional flow through the bioreactor.

In certain embodiments, a system of gravity perfused bioreactors can be readily enclosed in a secondary containment enclosure.

In certain embodiments, a system of gravity perfused bioreactors uses tilted supply and collection reservoirs or bags to minimize height differences during prolonged perfusion while enclosed in a secondary containment enclosure.

In certain embodiments, a system of battery-powered pumped perfused bioreactors can be readily enclosed in a secondary containment enclosure and operated for several days without intervention.

In certain embodiments, a perfusion system accommodates both passive (gravity-fed) and active (mechanical or other active pumping) perfusion.

In certain embodiments, a perfusion system accommodates integrated (on-board, self-contained) gravity-promoted flow and also accommodates external pressure or suction sources (such as mechanical pumps or external gravity-feed/collection reservoirs).

In certain embodiments, a gravity-fed perfusion system allows flow rate and pressure to be adjusted on-the-fly by changing the relative pressure differential between supply and collection reservoirs by adjusting the relative elevation of supply fluid to collection fluid. This may be accomplished by changing perfusate volume in the reservoirs or elevating the supply reservoir relative to the collection reservoir.

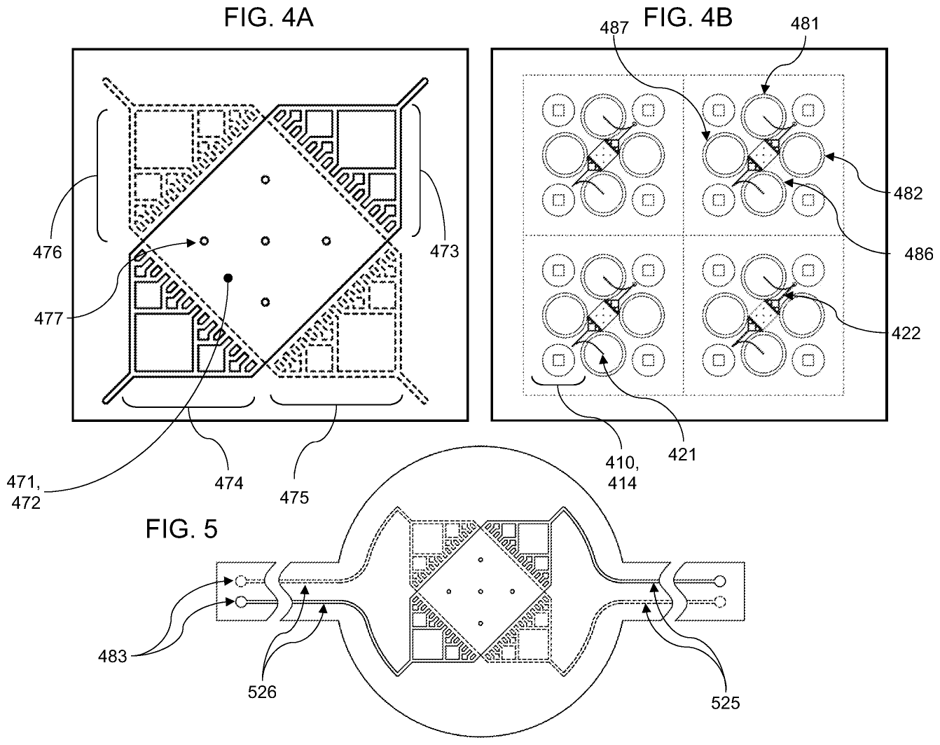

These and other aspects of the invention are further described below. Without intent to limit the scope of the invention, exemplary valves, bioreactor systems, methods and their related results according to the embodiments of the invention in conjunction with the accompanying drawings in FIGS. 4A-31C are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

Noting the parallel between the design requirements of NVU, gut, and airway barriers, for the remainder of this description we will for simplicity and clarity focus our embodiments on an airway model, but the design features and principles presented herein will apply to other organ chips as well. FIGS. 4A-4B present several airway-on-a-chip designs that embody some of the key features of this invention. FIG. 4A shows the mask layout for an airway chip device. Endothelial (solid lines) and epithelial (dashed lines) networks are topologically and geometrically identical, such that both physical layers can be produced with a single mask. By symmetry, the PDMS layer that contains, for example, vascular (endothelial) fluid delivery network 473, vascular chamber 471, posts 477, and vascular (endothelial) collection network 474 on one side of the membrane would register perfectly with the same layout applied to airway (epithelial) fluid delivery network 475, vascular chamber 472, posts 477, and airway (epithelial) collection network 476 on the opposite side of the membrane.

FIG. 4B shows a mask layout that includes reservoir footprints and connecting channels, with locations for vertical-via rotating valve actuator 410, vertical-via rotating valve 414, reservoir-to-valve channel 421, valve-to-bioreactor channel 422, supply reservoir vascular side 481, supply reservoir airway side 482, collection reservoir vascular side 486, and collection reservoir airway side 487. FIGS. 4A and 4B demonstrate that the fluidic channel designs we present can be mass produced in large quantities by either hot embossing, replica casting, or injection molding.

Following the design concept shown in FIGS. 4A-4B, FIG. 5 presents a common mask for the perfusion network for both chambers of a two-chamber, ribbon-puck bioreactor such that 180 degree rotation of the mask can be used to create two complementary fluidic networks in a barrier bioreactor, with input channels 525, output channels 526, and external connection ports 583, another demonstration of the simplicity and manufacturability of the subject devices.

A requirement for perfusion of the airway chip and other universal gravity-fed bioreactors is the ability to adjust the flow rate through each layer of a two-chamber bioreactor independently. One of the challenges with gravity-fed bioreactors is that there are only two ways to adjust the flow rate through the bioreactor: by controlling the height difference between the supply and collection reservoirs, and by adjusting the channel width in the device to reduce or increase the hydraulic resistance of the fluid network connecting the two reservoirs. The former is straightforward but tedious and may limit the dynamic range over which the reservoir heights can operate. The latter involves the fabrication of a new chip and allows a chip to have a single flow rate, rather than different flow rates at different times, e.g., high flow rates for cell loading and low flow rates for perfusion. To provide the capability to control fluidic resistance without fabricating a new chip, we present a very simple mechanical valve that can be used to regulate the hydraulic resistance without physically modifying microfluidic chip or the bioreactor therein.

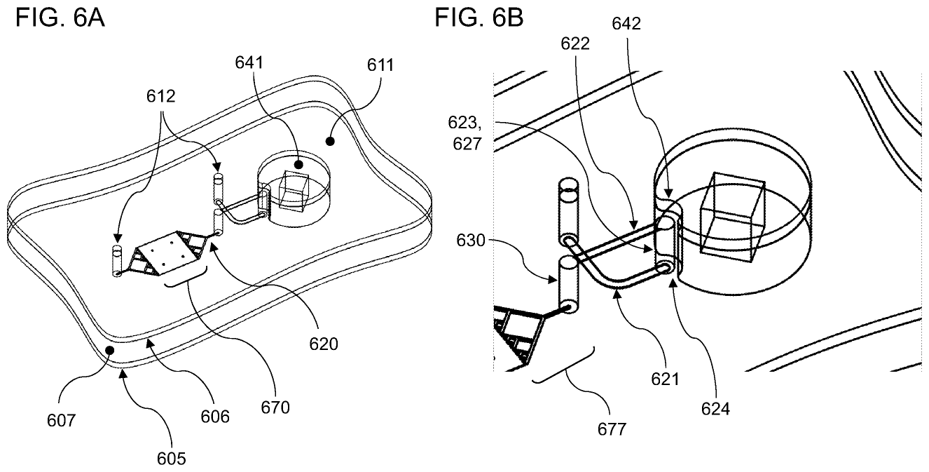

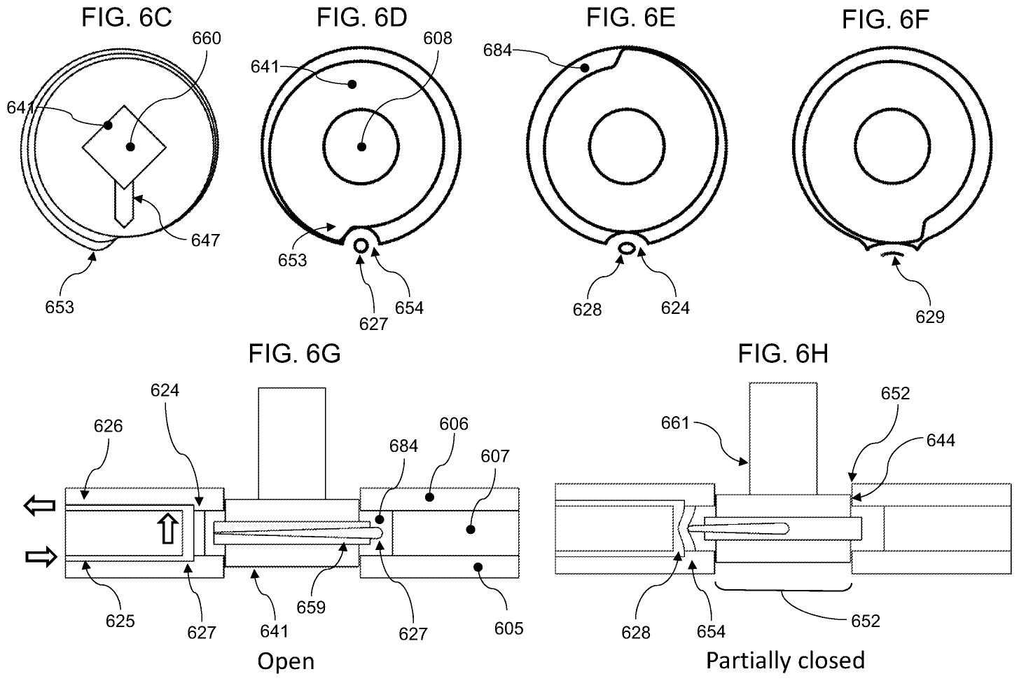

FIGS. 6A-6H provide details of a vertical-via rotary valve (VVRV) used in conjunction with a microbioreactor, in which the flow through the bioreactor is controlled by rotating a simple vertical-via rotary valve actuator. FIG. 6A is an isometric view of the assembly showing access port 612, vertical vias 623 and 630, planar channel network, lower and upper membranes 605 and 606, respectively, and actuator 641.

FIG. 6B provides a detailed view of channels, vias, and actuator in fully open position in a selected region in valve body 611. Port 612 provides a port in the valve body for external tubing connection. The fluidic channel in valve body 620 connects a vertical via 630 to the bioreactor 670. The valve provides a controlled connection between reservoir-to-valve channel 621 and valve-to-bioreactor channel 622, with vertical via 623 being adjacent to VVRV actuator 641. The wall 624 between vertical via and actuator separates the controlled via 623 from the actuator and will be compressed or not depending upon the angular orientation of the actuating trough 642 in the body of the VVRV actuator 641. Given that the vertical via 623 is punched or molded through the PDMS slab 607 that forms the central section of the three-part VVRV body 611 and that the input or output channels from the valve 621 and 622 will be on opposite sides of the valve body 611, a vertical via 630 is used to return fluid from the valve to the lower layer where via 630 connects to channel 620 in the lower level of the assembly 611 and hence to bioreactor fluidic network 677 and bioreactor chamber 670.

Note that the channels on both sides of 611 can be embossed or molded into either the upper and lower surfaces of the middle slab 607 or in the lower surface of the upper membrane 606, or the upper surface of the lower membrane 605, or combinations thereof. The three layers, 605, 607, and 606, should be bonded to each other, either by thermal or chemical means, to make the channels leak-tight. Note that in this device, the fluid-carrying channels do not make direct contact with the actuator 641, whereas in prior art shown in FIG. 2D, the fluid-carrying channel within the valve is actually a part of the actuator, such that fluid may be leaked into or trapped within the space between the valve body and the actuator, thereby presenting a risk of contamination within the bioreactor or leakage to the outside.

In the situation shown in FIG. 6B, the vertical via 627 is open because of the orientation of the actuator 641 in the body of a vertical-via rotating valve 611. In this embodiment, the shape of the actuator groove 642 compresses the full length of the via and the upper and lower layers 606 and 605, thereby applying strain to the bond between layers. Alternatively, the region where the via is compressed can be restricted to the central region in the middle layer 607, thereby reducing the required compressive forces on the chip and localizing them to a region where there are no layer-to-layer bonds. Furthermore, the shape of the actuating region can be adjusted to minimize frictional forces and torque requirements, as well as provide a larger angular range of via closure than would be possible with the embodiment in FIGS. 6A-6B.

The operating principle of the VVRV with a narrow actuating trough 642 is depicted in FIGS. 6A and 6B, which show a hard plastic actuator embedded in a three-layer elastomeric chip. Fluidic channels, which are embossed in the top surfaces of the lower membrane and the middle layer, are connected vertically with vias, and capped by the layer above or below each. The actuator, which may be rotated about its vertical axis, features a vertical trough which, when aligned with the via, un-pinches it and allows flow. As the actuator is rotated from that position, the via is pinched closed and flow is restricted.

The key feature of the trough is that it provides an actuator with different radial distances from the axis of rotation: the deepest part of the trough is designed so that a vertical via adjacent to that spot will be open. Outside the trough, the radius of the actuator is such that when a vertical via is adjacent to a region with the larger radius, the via will be compressed and sealed. As shown in FIG. 6B, the via will be open for a very small range of angles of rotation of the actuator and open for the rest. Alternatively, the trough could be widened to encompass almost all of the actuator circumference for the opposite effect, with the valve being closed for only a small range of actuator angles and open for the rest. In this case, the actuator could be described as having a lobe or a protrusion rather than a trough, but the operating principle is the same. The angular dependence of the opening or closing of a VVRV is thereby determined by how the radius changes as a function of angle from the open value to the closed one, with the possibility of intermediate values being useful for metering or mixing.

The operating principle of the VVRV with a tapered actuating protrusion is depicted in FIGS. 6C through 6F. FIG. 6C provides key details of the actuator 641: drive recess 660 to control the angular orientation of the actuator in the valve body, actuator position indicator 647, and actuator protrusion 653. FIGS. 6C-6H demonstrate the operation of the VVRV. In FIG. 6D, the wall 654 between vertical via 627 and actuator 641 is uncompressed because of the location of the actuator protrusion 653. In FIG. 6E, the actuator has been rotated by VVRV actuator drive axle 608 so that the via wall is partially compressed (624), while in FIG. 6F, the actuator is rotated to completely close the via 629.

FIGS. 6G-6H show cross-sectional views of the VVRV in operation. As shown in FIG. 6G, input channel 625 is connected to output channel 626 by open vertical via 627. The actuator protrusion 653, extending beyond via-valve actuator disk 659, is not in contact with the via wall 624 but instead is in the recess 684 in the valve body to clear the actuator protrusion in via-free region. The partially closed vertical via 628 shown in FIG. 6E and the totally closed vertical via 629 shown in FIG. 6F illustrate how the tapered protrusion affords gradual control of the via's open cross-sectional area, thereby regulating the flow.

The actuator 641 is enclosed by the body of a vertical-via rotating valve comprising layers 605, 606, and 607. The actuator cylindrical bearing surface 644 maintains the alignment of the actuator 641 in the hole 652 in the valve body to allow precise, low-friction rotation of the valve actuator. The compression region 654 is deflected by the actuator protrusion 627 to partially close the via 628. A drive shaft 661 for the actuator for the vertical-via rotating valve controls the angle of the actuator and its protrusions relative to the via.

FIGS. 6G-6H demonstrate one of the important features of this valve in contrast to the TURN valve shown in FIGS. 2A-2C: the forces delivered to the elastomer by the actuator are radial and do not attempt to lift the PDMS away from the underlying substrate.

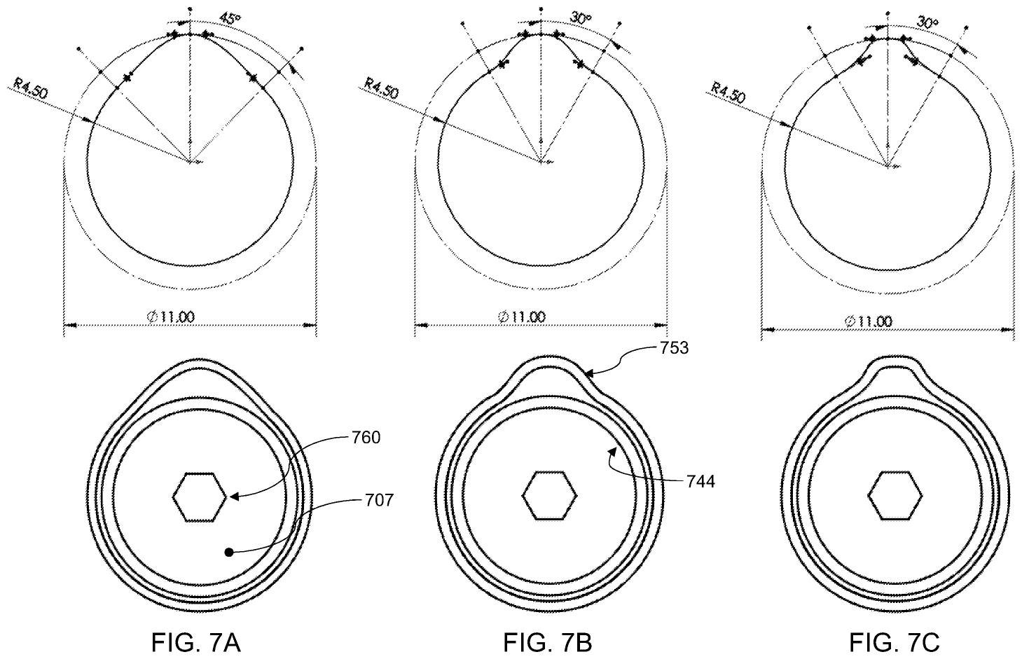

Another advantage of the vertical-via rotary valve is that the configuration of the actuator protrusions can be adjusted without having to alter the fluidic chip and its microfluidic channels and vias. For example, as shown in FIG. 7A-7C, protrusions 753 out from the valve actuator body 707 may be configured to suit specific needs of the corresponding vertical-via rotary valve (VVRV). For example, the topography of protrusion 753, the angle at which protrusion 753 approaches/departs via 623 as the actuator is rotated while constrained by bearing surface 744, and the circumferential shape of protrusion 753 may be adjusted. Drive recess 760 may also take various forms. FIG. 7A-7C detail select embodiments of the actuator body 707.

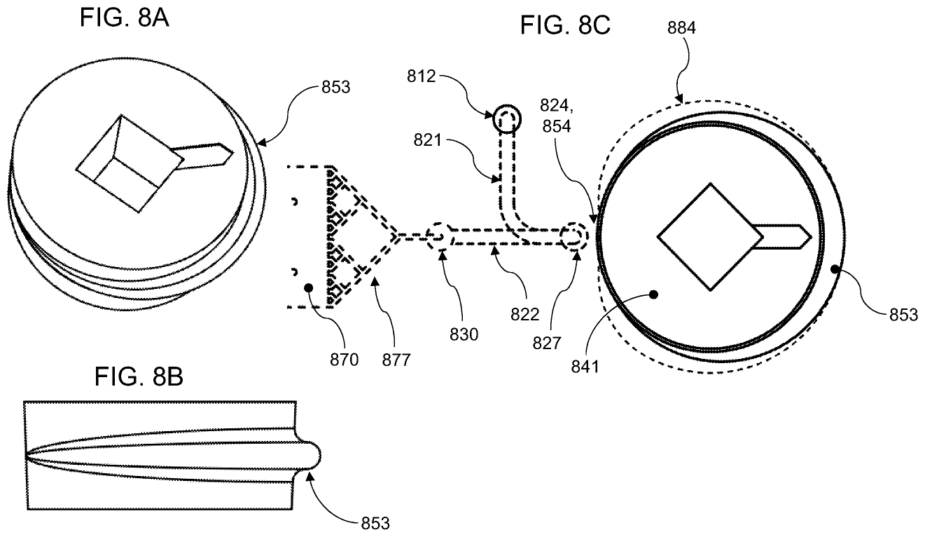

Referring to FIGS. 8A-8C, a vertical-via rotary metering valve is shown according to one embodiment of the invention. While the valves in FIGS. 2D and 2E, as well as classic microfluidic pneumatic valves, exhibit binary, i.e., on-off control of fluids, the TURN valve in FIG. 2A-2C allows for graduated adjustment of the closure, with an accuracy limited by the pitch of the adjusting screw and the compliance of the PDMS. In contrast, the ability to shape the actuator protrusion 852 in FIG. 8 allows us to create a vertical via rotary metering valve. FIGS. 8A and 8B show isometric and profile views of the metering actuator and its eccentric, protruding cam 853. FIG. 8C depicts the metering actuator as used in an exemplary valve body, in fully open position. Note the reduction near the via in radial depth of the recess 884 in the valve body that clears the actuator protrusion (cam) in the via-free region.

This third embodiment of the VVRV was designed with precision adjustment of flow rate in mind. We call this version the vertical-via rotary metering valve. The radial protrusion of the cam gradually increases from zero, i.e., open to closed, as the actuator is rotated up to 180°, allowing the user to fine-tune the magnitude of occlusion seen by the via. FIG. 8C shows how the selected region in valve body 811 contains a port for external tubing connection 812, a reservoir-to-valve channel 821, and a valve-to-bioreactor channel 822. As drawn, the wall 824 separates open vertical via 827 and the rotating actuator 841. The vertical via 830 returns the fluid to the lower layer. The action of the valve occurs in the compression region 854 that engages the actuator protrusion, thereby controlling the delivery or removal of fluid from bioreactor chamber 870 via bioreactor fluidic network 877.

It should be clear that the compressive forces that close the via are radial, do not apply a separating force directly to a bonded layer, and provide continuous metering whose angular dependence can be specified by the design of the protruding cam.

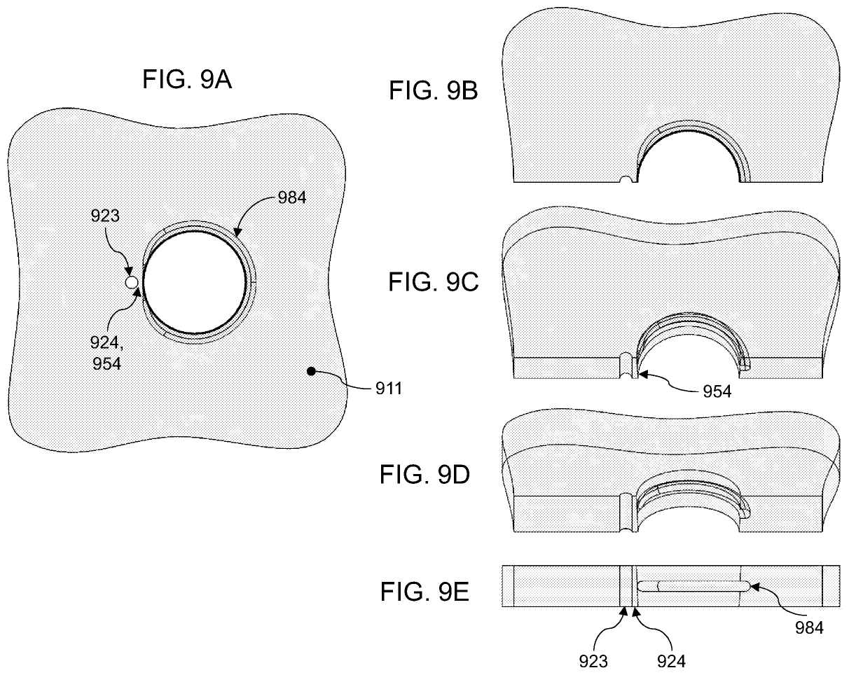

As shown in FIGS. 9A-9E, an embodiment of the vertical via rotary metering valve in which recess 984 provides clearance for protrusion 853 (or similar) in actuator 841 (or similar) to be present within valve body 911 without deforming valve body 911 except in compression region 954. When protrusion 853 overlaps with compression region 954, wall 924 is deformed toward via 923, decreasing the cross-sectional area of via 923, thereby throttling passage of fluid through via 923 to a degree proportional to the radius of protrusion 853 in the region of compression region 954. FIG. 9A depicts a selected region of valve body 911 as viewed from above. FIGS. 9B-9E show section views of valve body 911 from different perspectives.

The merit of this design is that the recesses shown in FIGS. 9B-9E can all be formed by injection molding or replica casting with a mold that is the negative of the specified shape that can be readily removed because the valve body is a flexible elastomer that can be stretched to remove the mold and insert the actuator. Alternatively, the actuator could in fact be used as the mold, as long as the elastomer does not stick to the actuator during the casting process.

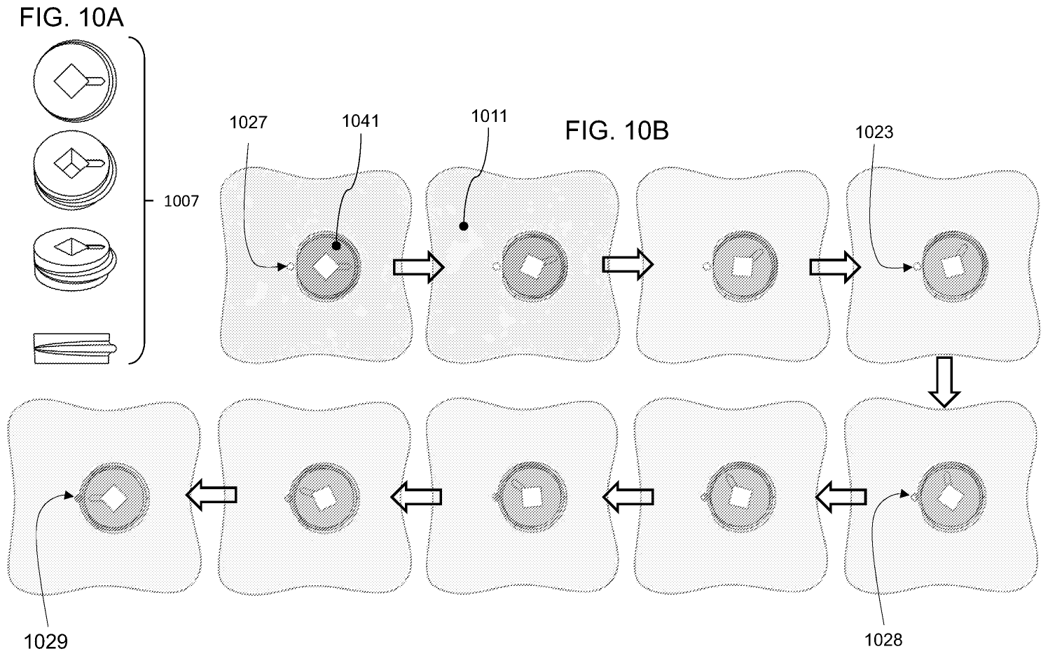

FIGS. 10A-10B depict a actuator body 1007 as used in the vertical-via rotary metering valve body 1011, and describes valve assembly and function. FIG. 10A shows views of the actuator body 1007 from different perspectives. As shown in the plan view in FIG. 10B, the actuator body 1041 is shown rotating counterclockwise about its axis such that protrusion 853 deforms wall 924 in compression region 954, closing via 1023 to an increasing degree. While protrusion 853 is not present within compression region 954, fully open via 1027 does not constrict fluid flow. As protrusion 853 enters compression region 954, partially closed via 1028 begins to throttle fluid flow therethrough. Finally, when protrusion 853 deforms wall 924 to the maximum extent, fully closed via 1029 completely blocks flow therethrough.

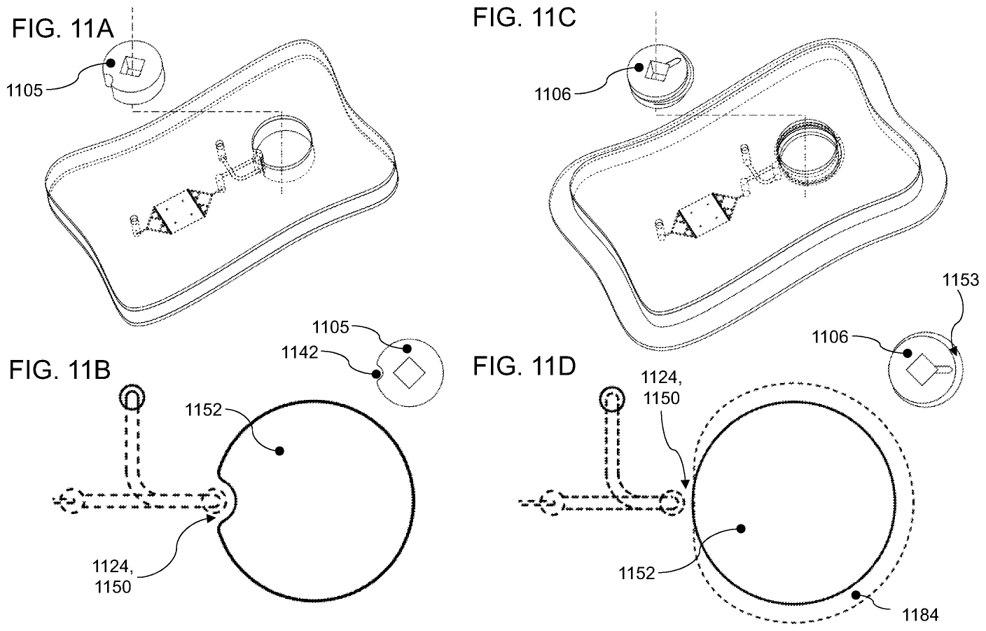

In a comparison of the operation of different actuator designs that we have presented, FIGS. 11A-11D show exemplary actuator embodiments 1105 and 1106 as used in conjunction with via and channel layer embodiments. Note that the deepest part of the trough in FIGS. 11A-11B serves the same via-open function as the portion of the circumference of the actuator in FIGS. 11C-11D without any protrusion

FIGS. 11A-11B describe the function of actuator 1105, which resides in cavity 1152 and provides a fully open or fully closed state of via 623, depending on the presence of actuator trough 642 within compression region 1150, as shown in FIGS. 6A-6B. In this embodiment, via wall 624 protrudes into valve cavity 1152. When actuator trough 642 aligns with compression region 1150 as shown in FIG. 11A, wall 624 remains undeformed and via 623 remains fully open. When actuator 1105 is rotated to a position in which trough 1142 does not align with compression region 1150, wall 1124 is fully deformed such that via 623 in FIG. 6B is pinched closed and blocks fluid flow therethrough. The present embodiment may require a middle slab, an upper layer, and a lower layer (shown in FIG. 6G), each of which is constructed of an elastomeric material, which may be mounted to a different substrate such as a glass slide. The upper and lower channels depicted may be in the middle layer, membrane layer(s), or a combination.

In FIGS. 11C-11D, metering actuator 1106 with raised protrusion 1153 is used in cavity 1152 to provide adjustable flow through via 1023. Recess 1184 provides clearance for protrusion of actuator 1106 to be present within the valve body without deforming the valve body except in compression region 1150, thereby reducing the PDMS distortional and frictional forces encountered upon rotating the actuator. In this embodiment, the actuator lobe protrudes into via wall 1124 when actuator 1106 is oriented accordingly. As actuator 1106 is rotated such that its protrusion deforms the via wall, fluid flow through the via is restricted to a degree proportional to the radius of protrusion into the compression region 1150. The construct pictured requires a middle slab, an upper layer, and a lower layer. Upper channels may be in the middle layer or the upper (membrane) layer, both of which are constructed of elastomer. Lower channels are in the middle layer in this embodiment, which requires a glass substrate.

While the valve body shown in FIG. 11A was designed specifically for the trough-actuator shown, the control of the open-closed state of the valved via shown in FIG. 11A could be accomplished with other actuator designs. The primary issue is to ensure appropriate control of the location of the actuator within the hole in the valve body. The actuator 1106 shown in FIG. 11C accomplishes this with the cylindrical bearing surfaces above and below the protrusion, as also shown in FIGS. 6G and 6H.

Another important aspect of this design is that it can scaled to different sizes; the actuators in current designs are 10 mm in diameter; larger and smaller designs could be readily implemented.

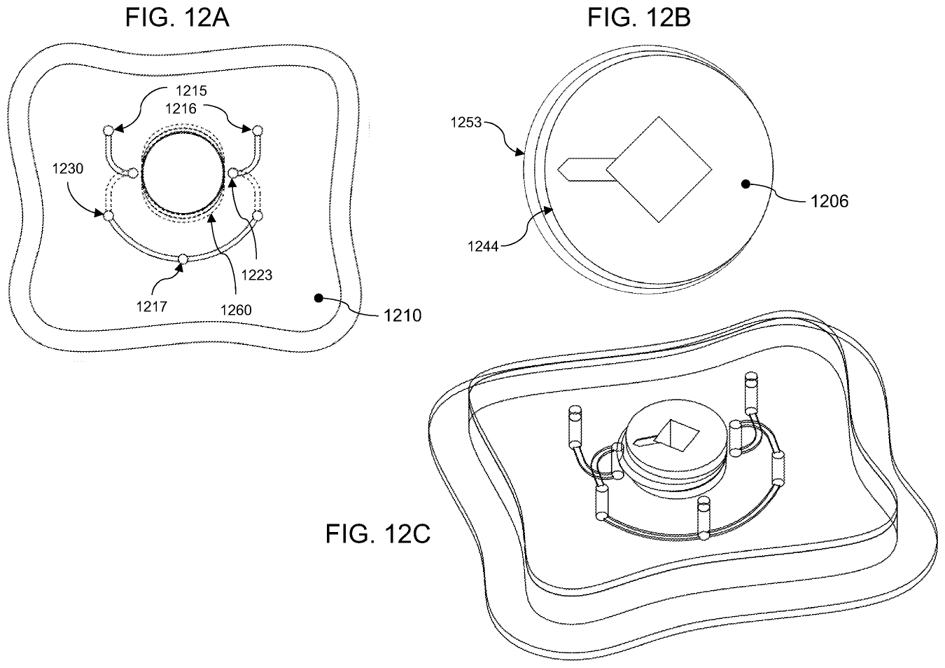

We have identified a further application of the vertical-via rotary metering valve concept, which is to incorporate multiple vias whose flow rates are controlled by a single metering actuator. FIGS. 12A-12C demonstrate how the vertical-via rotating valve concept can be immediately applied to create a vertical-via rotary valve that mixes two solutions in a user-controlled ration, just as is done in a modern hot-cold sink faucet: two inputs, one output, and the ability to select in real time and control the ratio of the two inputs.

One embodiment of this approach is shown in FIGS. 12A and 12C, which show a valve body 1210 with two inlet ports 1210 and 1211, each connected in series to an actuated via 1223 and then a non-actuated via 1230, with the two paths converging into a single outlet port 1260. The metering actuator 1206 shown in FIG. 12B would be inserted into the valve body of FIG. 12A. In the position shown, Inlet 1 would be at maximum occlusion while Inlet 2 would be at maximum flow. As the actuator is rotated, the ratio of solution 1 flow rate to solution 2 flow rate begins to invert, until Inlet 2 is at maximum occlusion and Inlet 1 is at maximum flow (at actuator rotation=180° from that pictured). As a result of this design, the relative flow rates of input solutions 1 and 2 are adjusted by a rotating metering actuator, allowing relative concentrations to be adjusted. Note that with a different shape for the actuating protrusion, it would be possible to have different, non-linear scaling of the relative contributions of the two sides.

The embodiment shown in FIGS. 12A-12C may require an internal mold to cast the recess 1260 for the valve actuator protrusion. The elasticity of the valve body should be sufficient to remove the mold. If not, the mold can be separated into components that can be removed individually. The vias could be punched, but it would be preferable to have them cast in place during the molding of the valve body to ensure verticality and correct position and diameter, and to avoid the depth-dependent distortion of the punched hole.

To the inventors' best knowledge, no other microfluidic valve that with a single actuator can provide such mixing.

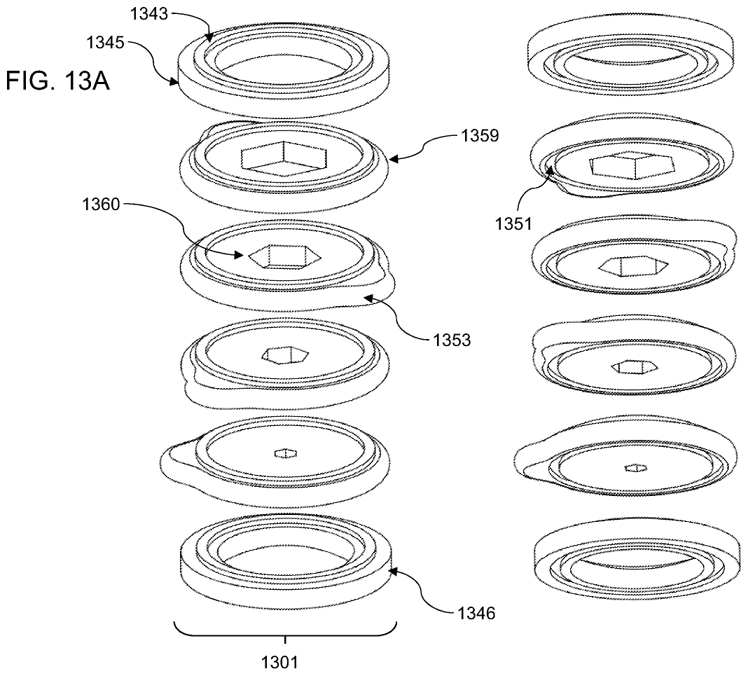

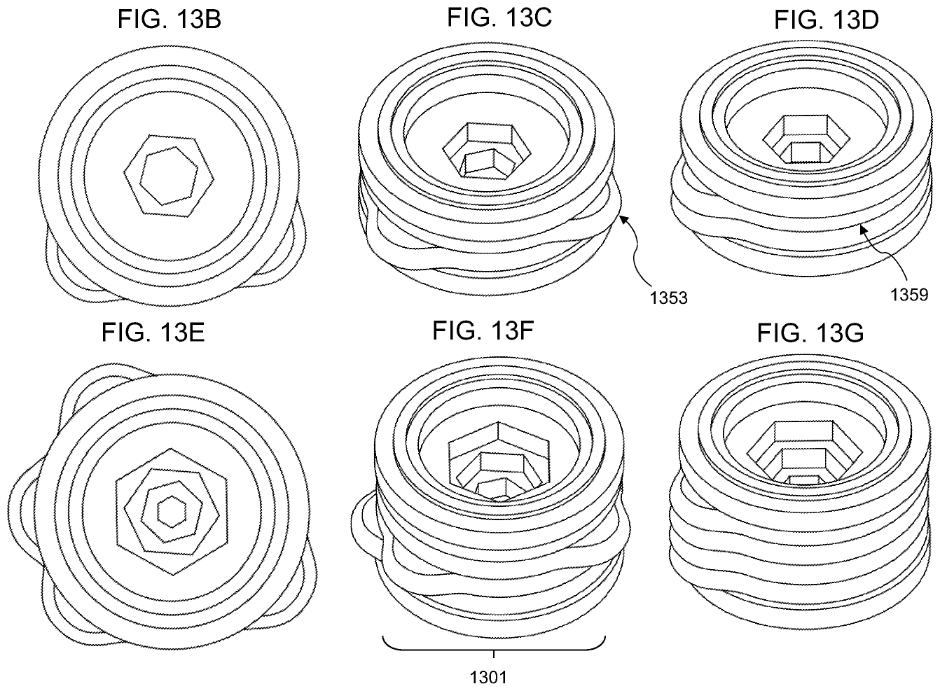

Independent control of fluid flow through multiple vertical vias on a single chip may be accomplished using an embodiment of independent coaxial actuator assembly 1301 shown in FIGS. 13A-13G, provided that the valve body has adequate thickness to afford enough via height for multiple stacked actuators.

FIG. 13A depicts an exploded view of a stack of actuator discs 1359 capped on top and bottom by bearings 1345 and 1346, respectively. Alignment ridges 1343 on bearings 1345, 1346 and discs 1359 mate with alignment grooves 1351 in the adjacent actuator disk or bearing such that mutual axial alignment of all elements is ensured. Each of the actuator discs 1359 may be rotated independently from any other disc 1359, thereby allowing that disc's protrusion 1353 to address the desired vertical via within the valve assembly. In the present embodiment, each actuator disc 1359 features drive recess 1360 to facilitate rotation by inserting an appropriate tool into recess 1360 and turning the tool.

FIGS. 13B-13D show one embodiment of independent coaxial actuator assembly 1301 that features two actuator discs 1359 in various independent orientations. As previously described in this invention, protrusions 1353 can be positioned relative to the valve assembly's compression region so as to adjust the degree of via constriction.

FIGS. 13E-13G show one embodiment of independent coaxial actuator assembly 1301 that features four actuator discs 1359 in various orientations. As previously described in this invention, protrusions 1353 can be positioned relative to the valve assembly's compression region so as to adjust the degree of via constriction.

Alternatively, two coaxial actuator disks can have staggered surfaces such that the body of one is above the other, but the actuator lobe on each is coplanar, so that the two lobes of a single actuating plane can be adjusted independently. A locking screw can hold them at a fixed relative position. The embodiments shown herein are not meant to imply in any way a limitation in the arrangement and control of one or more actuator protrusions or troughs.

Other embodiments allow for a variety of different actuation configurations.

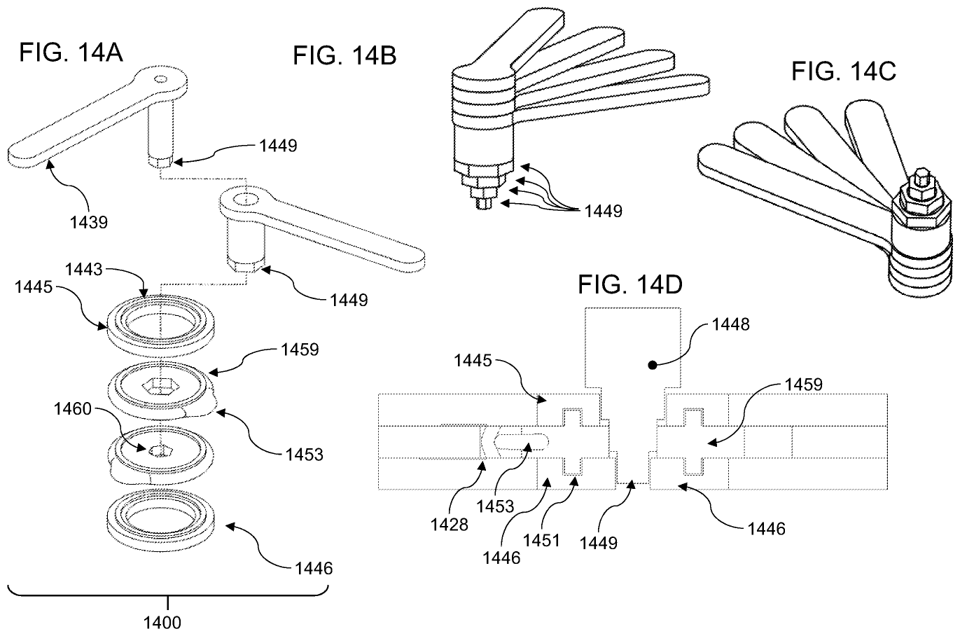

FIGS. 14A-14D delineate how one or more nesting wrenches 1439 could provide independent control of each actuator in actuator assembly 1400. A coupled wrench could provide coordinated actuation of a plurality of stacked actuators (FIGS. 14B-14C). The upper grooved actuator bearing 1445 and lower grooved actuator bearing 1446 act as rigid slip bearings to keep the 1448 shaft and actuator aligned in the elastomeric body. Actuator drive engagement spline 1449 may be a spline, D-shaft, square, or other non-rotating shape. A slip fit knob allows the user to remove the adjustment knob once the desired flow is chosen. At least one actuator handle 1439 with actuator drive engagement spline 1449 engages the drive recess 1460 in the via valve actuator disk 1459 to control the angular position of the actuator protrusion 1453. FIG. 14D shows the actuator knob 1448 and spline 1449 engaged with disk 1459 to control the compression of vertical via 1428 by the actuator protrusion 1453.

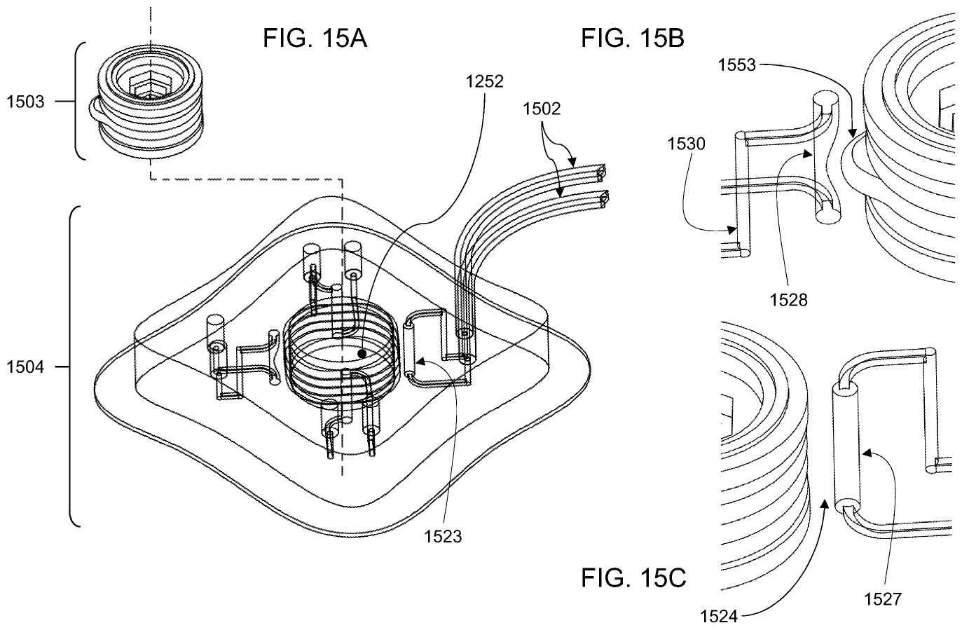

The concept of rotary actuators can be expanded by creating a stack of coaxial actuators that can each actuate one or more of a multiplicity of vias surrounding a common hole in the valve body, with each via being controlled independently. FIGS. 15A-15C detail the operation of independent coaxial actuator assembly 1503 when paired with valve assembly 1504. Valve assembly 1504 features a central hole 1252 with four isolated fluidic circuits, each containing two vertical vias—valved via 1523 similar to those previously described in this invention, and unvalved via 1530 that returns fluid to a lower level of assembly 1504. The use of unvalved second via 1530 to return the fluid back down to the lower fluidics layer allows a vertical pocket to support tubing 1502 that may connect valve assembly 1504 to reservoirs or off-chip bioreactors (some tubing removed for clarity), as shown in FIG. 15A.

As described elsewhere in this invention, actuator protrusions 1553 may be positioned relative to valved vias 1523 such that wall 1524 is partially deformed, in which case flow through via 1528 is throttled as in FIG. 15B; not deformed, in which case via 1527 remains unconstricted as in FIG. 15C; or completely occluded, in which case flow is completely prevented (not shown).

While this embodiment can have actuator recesses around the central cavity to keep the actuating disks in place, assembly of the valve would be simplified without them. It is important to realize that each via needs to be compressed by an actuator protrusion, and hence can either be configured as shown in FIG. 6D or FIG. 8C. Bearings or other mechanisms could keep the actuator in the proper vertical and horizontal position.

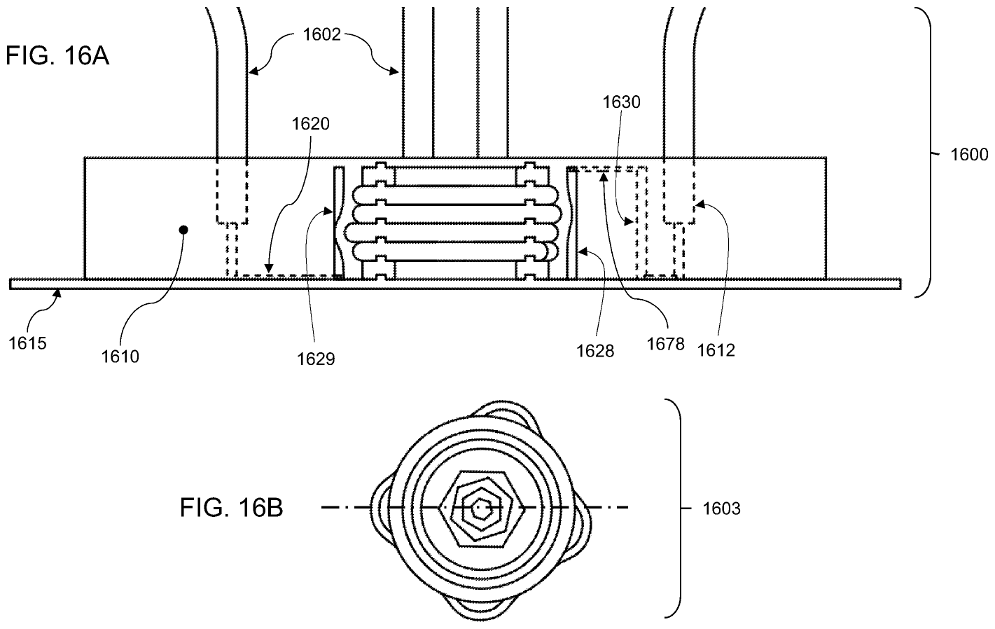

FIGS. 16A-16B present a cross-sectional view of an independent coaxial actuator assembly 1603 used with valve body 1610 as mounted on microscope slide substrate 1615. The via valve on the left has a fully closed via 1629, while on the right via 1628 is partially closed. Examining the figure in more detail, two isolated fluidic circuits can be seen in this view of valve assembly 1600, each circuit containing two vertical vias—valved via 1628, 1629, and unvalved via 1630 that returns fluid to a lower level of assembly 1600. In conjunction with horizontal channel 1678, the use of unvalved via 1630 to return the fluid back down to lower channel 1620 from the top of via 1628 allows port 1612 to support tubing 1602 that may connect valve body 1610 to reservoirs or off-chip bioreactors.

As described elsewhere in this invention, actuator protrusions may be positioned relative to valved via 1628 such that the wall between actuator 1603 and via 1628 is partially deformed, in which case flow through via 1628 is throttled; completely occluded, in which case flow through via 1629 is completely prevented; or not deformed, in which case the via remains unconstricted (not shown).

It is important to recognize that other than TURN or Twist valves, the vast majority of microfluidic valves are either open or closed, with little ability to throttle the output. TURN and Twist valves use a threaded screw to apply a downward force to close a horizontal channel. Overtightening the screw can debond the valve. The via valve in the present invention operates by means of a radial force that does not present a risk of valve debonding upon valve activation activation.

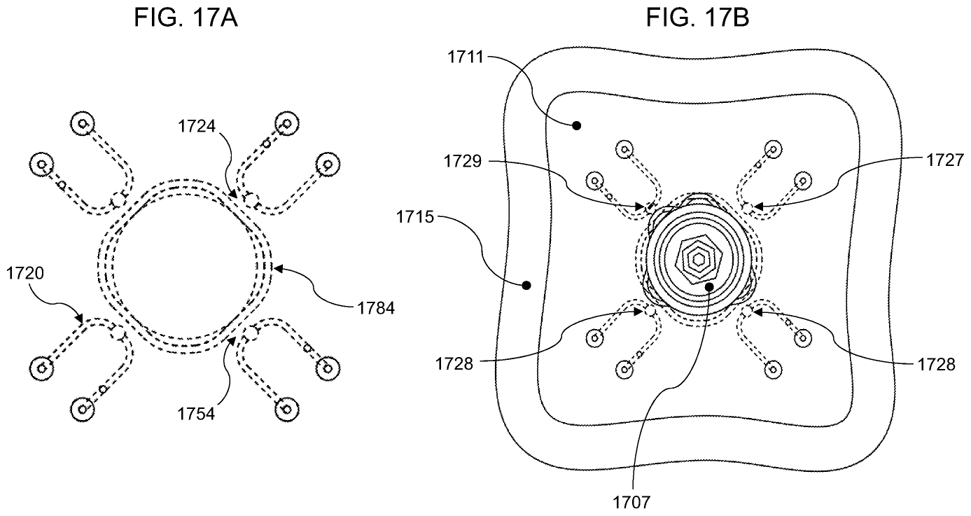

In a more detailed rendering of a four-channel vertical-via rotary valve, the actuator recesses that were not shown in FIGS. 16A-16B are shown as dotted lines 1784. FIGS. 17A-17B present an overhead view of the fluidic layout for a four-channel vertical-via rotary valve. Actuator assembly 1707 is used within valve body 1711 supported by glass substrate 1715, to control flow rate through channels 1720. As explained elsewhere in this invention, valve body 1711 features recesses 1784 that clear actuator protrusions in via-free regions. In compression regions 1754, the presence of actuator protrusions deforms walls 1724 into vias 1728 and 1729 to render them partially closed or fully closed, respectively. In the absence of an actuator protrusion within compression regions 1754, vias 1727 remain fully open to flow.

Note that in this illustration the compression of the elastomer between the fully closed via and the actuator is not shown in detail. In one exemplary embodiment, the actuator is similar to that shown in FIGS. 14A-16B.

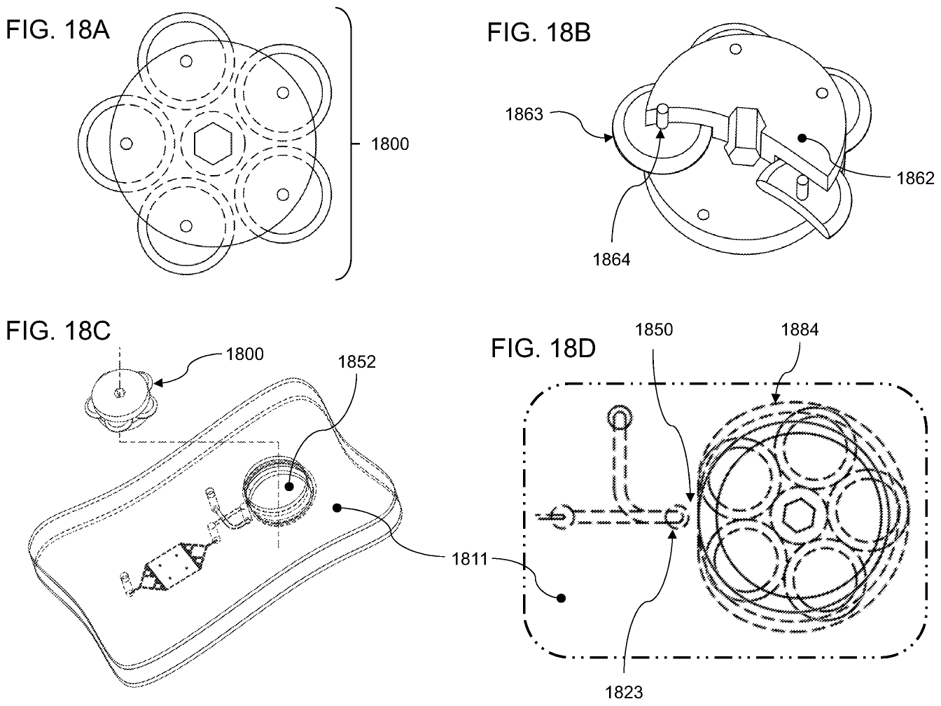

One of the limitations of the actuators shown so far is that the actuator protrusion has to slide across the PDMS surface in the vicinity of the controlled via such that the PDMS is compressed to close the valve. There will be friction associated with this sliding, which in turn requires application of a torque to overcome the frictional force. An alternative to this approach is to use the roller actuators shown in FIGS. 18A-18D.

This embodiment of the VVRV uses planetary actuator 1800 as shown in FIG. 18A. Actuator 1800 consists of five rollers 1863, central hub 1862, and roller axles 1864 that allow rollers 1863 to rotate but not translate relative to drive disk 1862. Actuator 1800 is installed into cavity 1852 in valve body 1811, and hub 1862 may be rotated relative to valve body 1811. Rollers 1862 are confined by matching recess 1884 in the elastomeric valve body 1811, which fixes actuator 1800 along the vertical axis. As actuator hub 1862 is rotated, rollers 1863 depart recesses 1884, arrive in compression zone 1850, and restrict flow through via 1823, as described elsewhere in this invention.

While the functional principle is the same as that previously presented, roller actuator 1800 was designed to minimize friction, as it only contacts the valve body in the immediate region of actuation 1850 and the extremities of those rollers opposite the via.

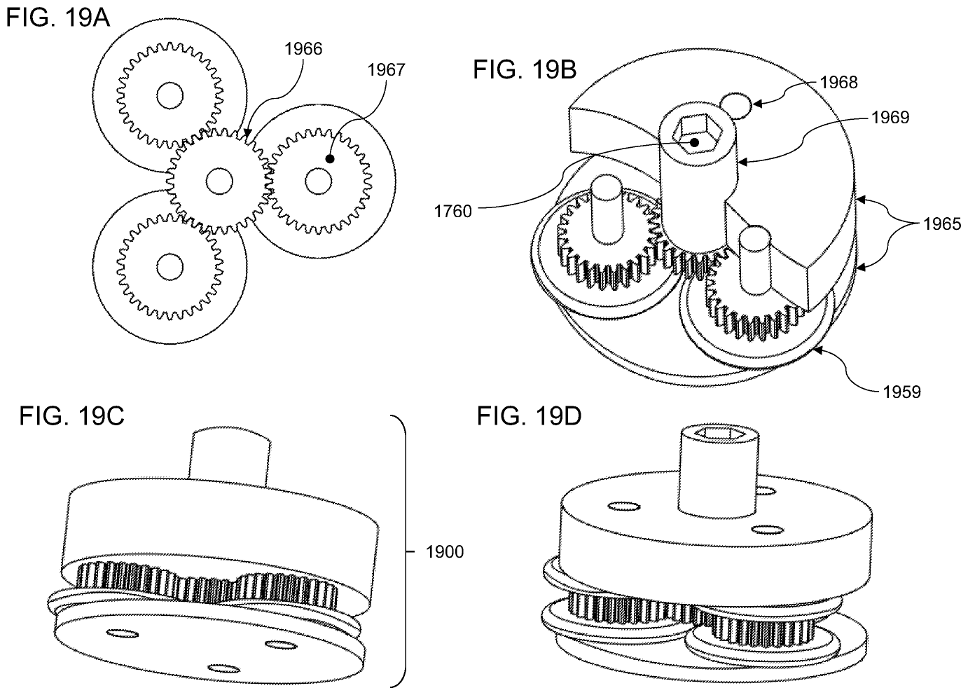

The torque required to rotate the roller actuator in FIGS. 18A-18D will be determined by friction in the bearings and whatever inelastic losses are associated with the rolling compression and decompression of the PDMS. One way to reduce the driving torque even further and provide a more precise control of the rollers is to drive them with sun and planetary gearing. FIGS. 19A-19D present planetary gear actuator assembly 1900, comprised of planetary gears 1967 that are affixed to valve actuator disks 1959 and axles 1968; sun gear 1966 that is affixed to drive shaft 1969; and rotating disks 1965 that are driven by axles 1968. When driven by a drive tool (not shown) engaged in the recess 1769, drive shaft 1969 transfers rotational motion to sun gear 1966, which in turn causes planetary gears 1967 to rotate. Planetary gears 1967 may rotate, but axles 1968 prevent planetary gears 1967 from translating relative to disks 1967. When actuator 1900 is used in a vertical-via rotary valve assembly similar to other actuators as described elsewhere in this invention, disks 1965 rotate relative to the valve body (not shown).

FIG. 19E shows another embodiment of a planetary actuator wherein planetary gears 1967, driven by sun gear 1966, rotate but do not translate relative to valve body 1911. Similar to other embodiments covered in this invention, gears 1966 and 1967 rotate about axles 1968 and 1969, respectively, but in the present embodiment disk 1955 is held stationary throughout operation. As explained elsewhere in this invention, depending on the extent to which actuator protrusions 1953 extend into compression regions 1954, vias 1927, 1928, and 1929 may be set to fully open, partially closed, or completely pinched off, respectively.

In summary, FIGS. 6A through 19E present different embodiments of a vertical-via rotary valve that can provide low-cost, on-chip, precise control of the flow of fluid through one or more microfluidic channels either as stand-alone valves or as components, for example, in a gravity-fed organ-chip system. This invention thereby enables the fabrication of general-purpose bioreactors whose flow rate could be adjusted after the device has been fabricated, and even while an experiment or measurement with these bioreactors was in progress.

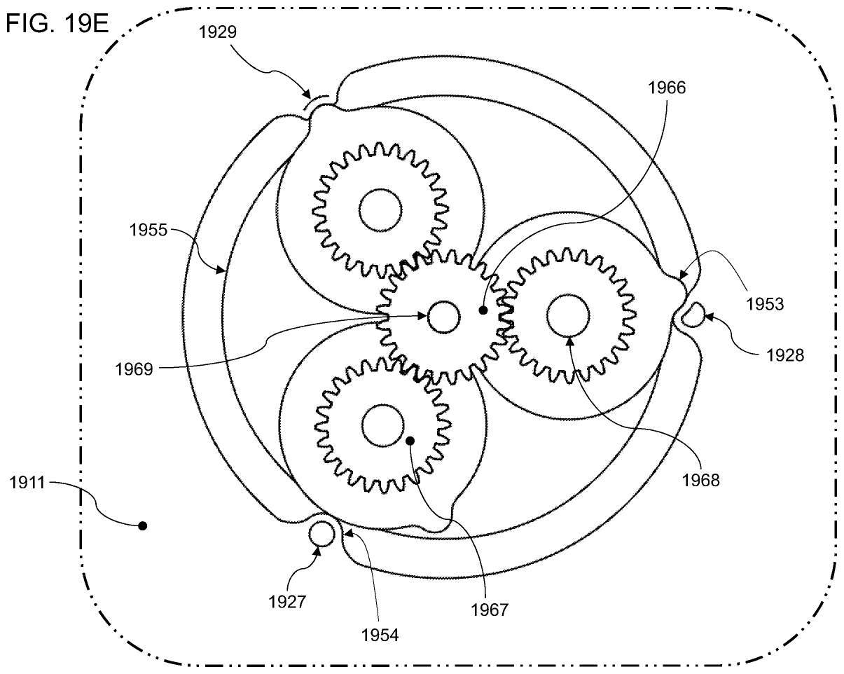

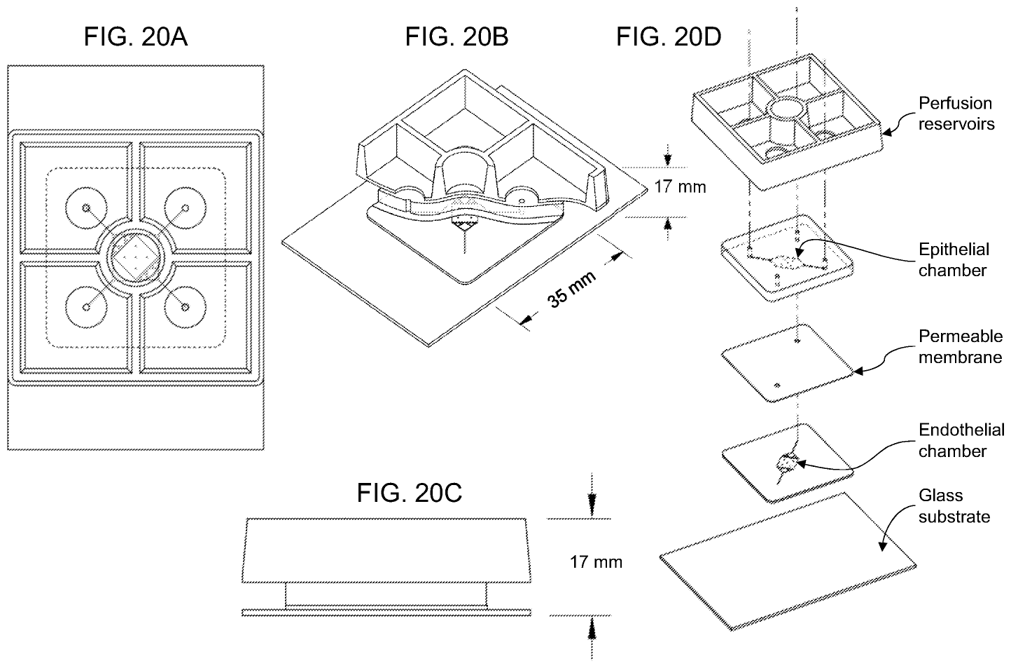

Referring to FIGS. 20A-20C, a gravity perfused bioreactor without a valve is shown according to one embodiment of the invention. FIG. 20A-20D show respectively a top view, a partially perspective view, a side view and an exploded vies of the gravity perfused bioreactor with the four reservoir chambers that support a single gravity perfused barrier bioreactor (without valves). For clarity of discussion, we will refer to this bioreactor as an airway model, but we have adapted its design from VIIBRE's neurovascular unit (NVU) to create an airway model, or lung-on-a-chip. The intention is to generate a microfluidic device in which both layers (endothelium, epithelium) can be perfused actively (with a pump) and passively (gravity-fed), depending on the stage of its employment. The passive perfusion option will be facilitated by on-chip reservoirs serving each of the four ports. These ports will also accept intubation from our standard Tygon tubing for active perfusion using syringe pumps or rotary planar peristaltic micropumps. The embodiment of the gravity perfused bioreactor shown in FIGS. 20A-20D is consistent with the design concepts presented in FIGS. 3A-3B.

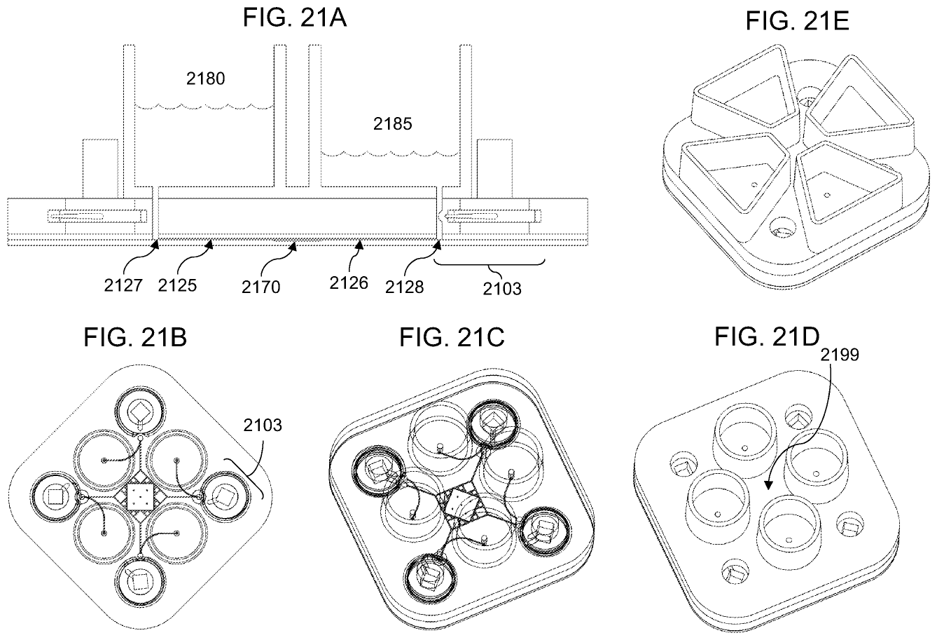

In recognition of the need to be able to adjust the perfusion rate of a generic gravity perfused bioreactor, FIGS. 21A-21E depict a gravity-fed bioreactor 2170 with integrated vertical-via rotary metering valves 2103 and various embodiments of on-board supply reservoirs 2180 and collection reservoirs 2185. Each reservoir 2180, 2185 is connected through via 2127, 2128, respectively, and perfusion channel 2125, 2126, respectively, to bioreactor chamber 2170. In the present embodiments, flow rate through the gravity-fed system is dependent on the height differential between supply reservoir 2180 and collection reservoir 2185. If desired, this system may also be used with active flow by connecting pumps to the ports in the floor of each reservoir. All actuators 2103 may be adjusted independently.

As shown in FIG. 21A, supply via 2127 is fully open while collection via 2128 is partially closed by corresponding actuator 2103. In the present state, the constriction of collection via 2128 results in decreased flow rate through and increased pressure within bioreactor 2170, supply channel 2125, and collection channel 2126.

FIGS. 21B-21D show a two-chamber bioreactor wherein each chamber is fed by a dedicated supply reservoir and analyte is retained in a dedicated collection reservoir. Actuators may be adjusted through access holes in the upper surface.

FIG. 21E illustrates a gravity-fed system with increased-capacity reservoirs. As shown in FIGS. 12B-12D, this embodiment features an observation window 2199 that offers unobstructed viewing of an underlying bioreactor or similar analytical chamber, as well as clearance for a microscope objective or other instrument.

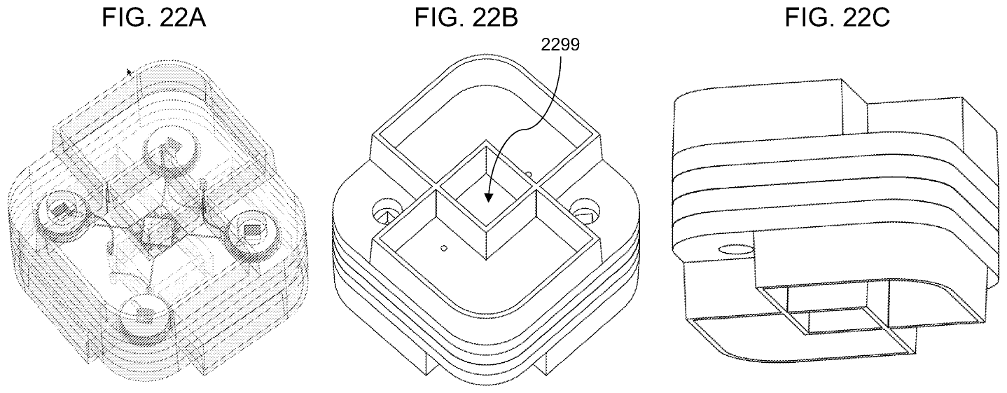

Given the need to seed cells on both sides of a barrier membrane while creating an airway chip, we have devised a bioreactor that has reservoirs on each side so that one side can be gravity perfused prior to cell attachment, at which point it is inverted and the other side is gravity perfused. FIGS. 22A-22C present an embodiment of an invertible, two-sided bioreactor with integrated vertical-via rotary valves and on-board reservoirs. FIGS. 22A-22B show isometric top views of the system, which has a pair of reservoirs—one supply reservoir and one collection reservoir—on the top surface, as well as an identical pair of reservoirs on the bottom surface as shown in FIG. 22C. Each pair of supply and collection reservoirs services one chamber of the bioreactor. Observation window 2299 is provided to allow an unobstructed view of the underlying bioreactor.

FIGS. 22A-22C show different views of a two-chamber bioreactor in which each chamber is fed by a dedicated upstream channel-reservoir pair, and is connected to a dedicated downstream channel-reservoir construct. As described elsewhere in this invention, flow rate through and pressure within each circuit is controlled by rotary-via valves located between each reservoir and its corresponding feed channel or collection channel.

The present system would be useful in an application wherein, for example, cells are to be seeded on a surface (e.g., a membrane separating the two bioreactor chambers) that will become a ceiling within the interior of the bioreactor chamber when the system is inverted.

Clearly, the present system can be used in gravity-feed mode only for the circuit connected to the upward-facing reservoir pair. However, as shown in FIGS. 22A-22C, any of the ports can be connected to a pump that actively perfuses the corresponding circuit.

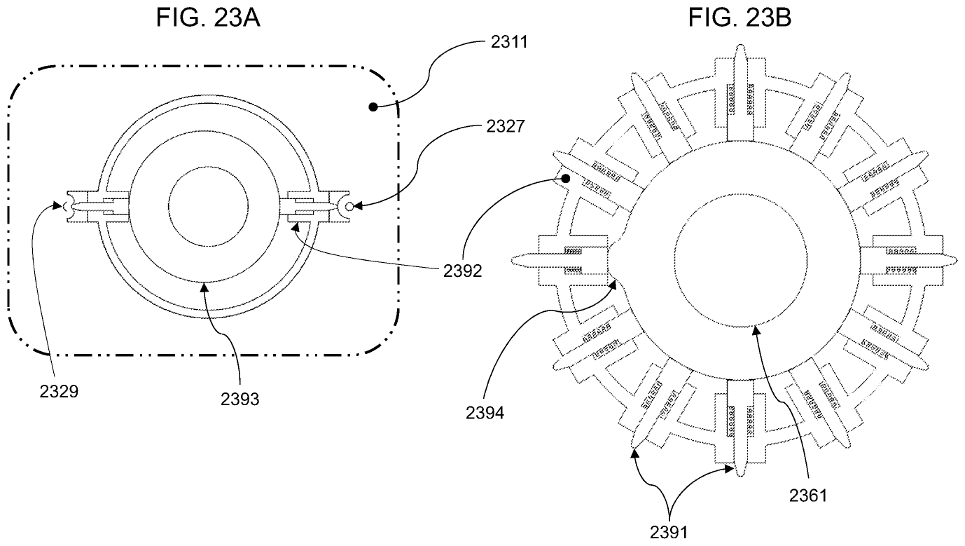

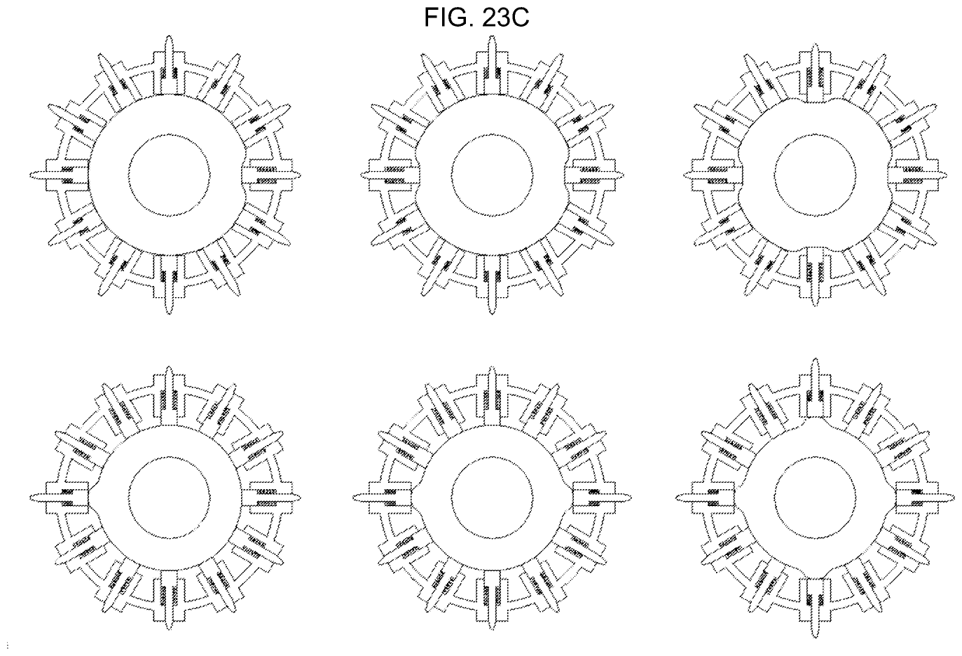

FIGS. 23A-23C present additional rotary valve designs that function under principles similar to those described elsewhere in this invention, with unique differences. In these embodiments, eccentric actuator cam 2393 or lobed actuator cam 2394 or similar is used to displace actuator pins 2391 toward or away from vias 2327, 2329, respectively. Actuator pin housings 2392 constrain movement of pins 2391 to their respective radial axes. As described elsewhere in this invention, deformation of the elastomeric walls separating actuator elements 2391 from vias 2327, 2329 alters the cross-sectional area of vias 2327, 2329, and thereby adjusts the capacity for volumetric flow rate therethrough. Actuator assemblies such as those presented here may be configured as modular mechanisms that can be inserted into valve body 2311.

FIG. 23A presents a two-via proportioning (mixing) valve, in which the constriction of via 2327 is inversely related to the constriction of via 2329. In the state depicted in FIG. 23A, via 2327 is fully open and via 2329 is completely closed to flow. As eccentric actuator 2393 is rotated by turning drive shaft 2361 from the pictured position, pin 2391 controlling via 2327 begins to push radially outward, and via 2327 begins to close, while pin 2329 begins to retract radially inward, causing via 2329 to start to open. If eccentric actuator 2393 is rotated 180° from the position shown in FIG. 23A, via 2327 would be fully closed, and via 2329 would be totally open. The degree of eccentricity and the radius or shape of cam 2393 may be modified to suit specific applications. The quantity and circumferential location of pin housings 2392 and pins 2391 may be adjusted.

In other embodiments of the spring-return-based valve, the surface of actuator cam 2393 that contacts pins 2391 may take various shapes, as illustrated by FIGS. 23B-C. For example, while single protrusion 2394 shown in FIG. 23B addresses pins 2391 one-at-a-time, in additional embodiments like those shown in FIG. 23C, protrusion 2394 may be expanded such that it displaces more than one pin 2391 at a time. Multiple protrusions 2394 may be configured to address pins 2391 in groups, as exemplified in FIG. 23C.

The spring force that returns pins 2391 from their extended positions to their relaxed state may be provided by the elastomeric material from which the valve body 3211 is constructed, as in FIG. 23A, or another spring-like construct may be incorporated to serve that purpose, as represented in FIGS. 23B-23C.

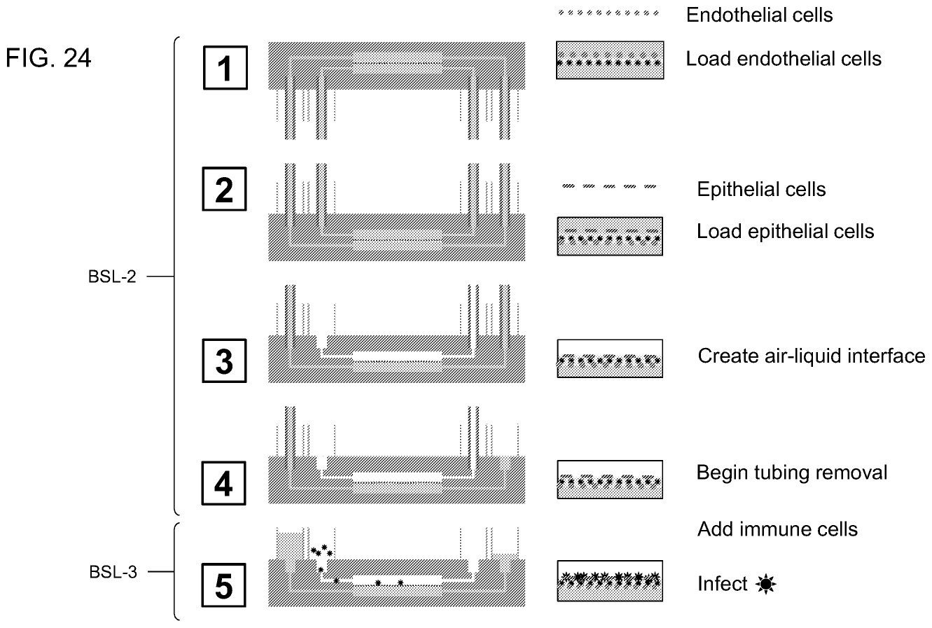

FIG. 24 delineates one possible sequence for seeding cells on both sides of an airway barrier membrane bioreactor that has reservoirs only on one side:

Step 1: Load endothelial side while inverted and maintain cells with pumped perfusion for one day. Both sides are perfused with pumps since this is not performed in BSL-3.

Step 2: Load epithelial side while right side up, maintaining both sides with pumped perfusion for one more day. Both sides are perfused with pumps as this is not performed in BSL-3.

Step 3: Remove tubing from inlet of epithelial side and pump to remove media from the entire airway chamber to establish a mature air-liquid interface.

Step 4: Invert the device with reservoirs up and seed the endothelial side. Perfuse with pumping until endothelium is mature.

Step 5: Remove tubing from outlet side of endothelial chamber to allow some perfusate to fill the tubing port.

Step 6: Remove the tubing from the inlet port and fill with required media for duration of experiment. Remove tubing from epithelial outlet.

It also may be possible to avoid the inverted tubing-perfusion steps as long as the cells in the lower chamber are immersed in trapped fluid without perfusion for only the several hour interval required to attach the cells on the other (then up) side of the barrier.

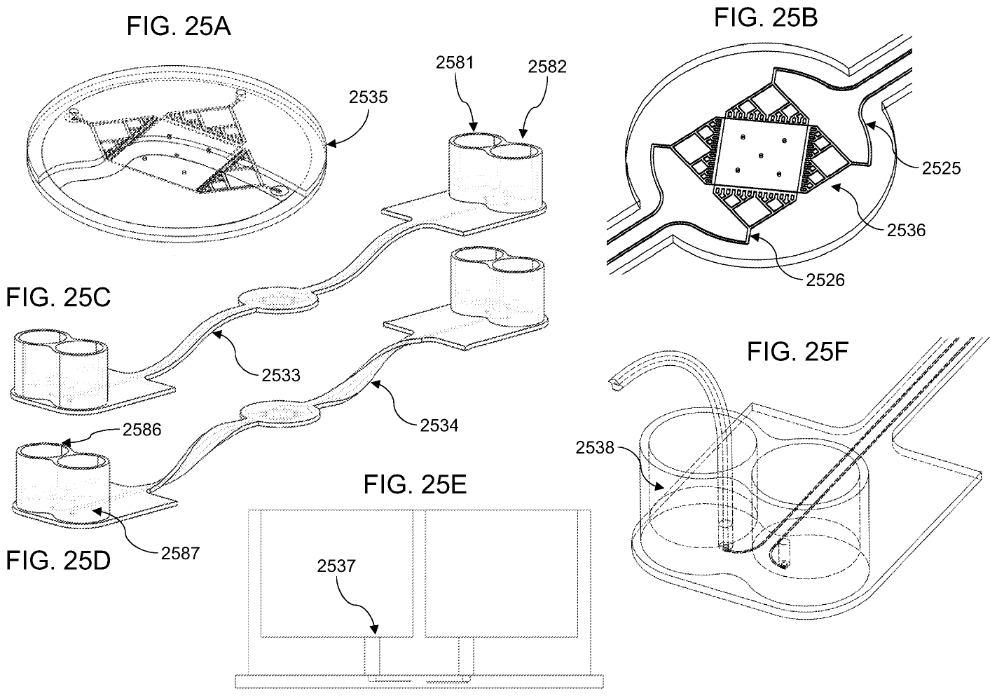

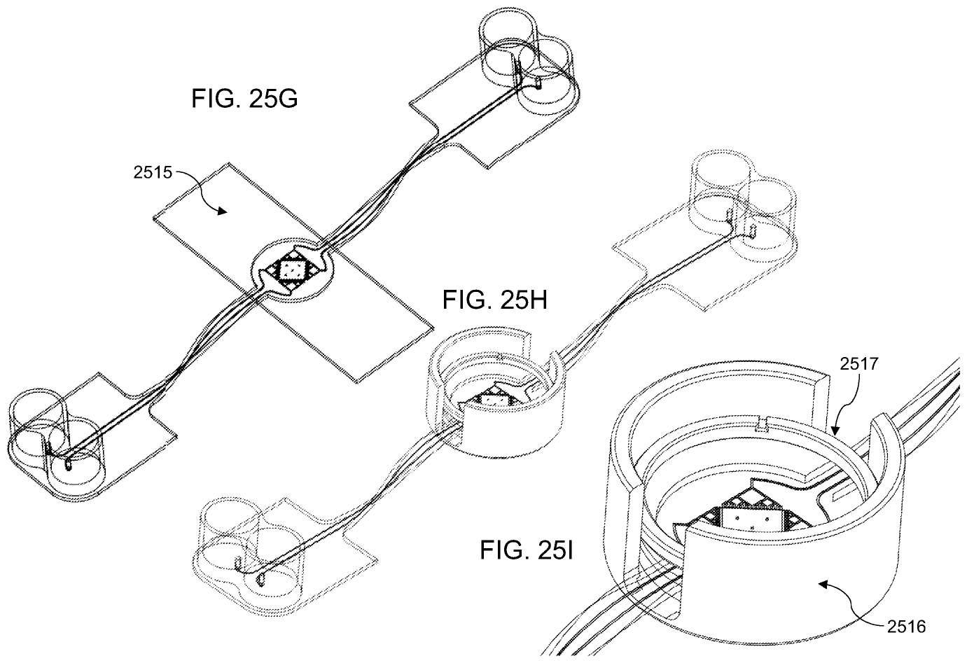

An alternative means to the inversion-for-cell-seeding step discussed above is to use ribbon fluidics to invert the bioreactor while leaving the reservoirs stationary. FIGS. 25A-25F present how this can be done using ribbon fluidics. FIG. 25A shows barrier bioreactor disk 2535 that can be bonded or otherwise attached to ribbon fluidic 2533, so that the bioreactor can be inverted, creating twisted ribbon fluidic 2534 that contains fluid manifold 2536 and supply and collection lines 2525 and 2526, respectively. FIG. 25E shows an intubation recess 2537 in the reservoir, and FIG. 25F shows an intubated reservoir 2538. Note that a generic perfusion manifold 2533 could be designed for use with a variety of bioreactor disks 2535, with attachment either by bonding or clamping as discussed below.

FIGS. 25G-25H present two different embodiments using gravity perfusion with ribbon fluidics for invertible loading. The supply reservoirs, the flippable ribbon airway bioreactor, and the collection reservoirs are terraced for passive pumping. FIG. 251 shows openable puck housing 2516 and openable puck compression ring 2517. In FIG. 25G the flippable airway bioreactor is mounted on microscope slide 2515.

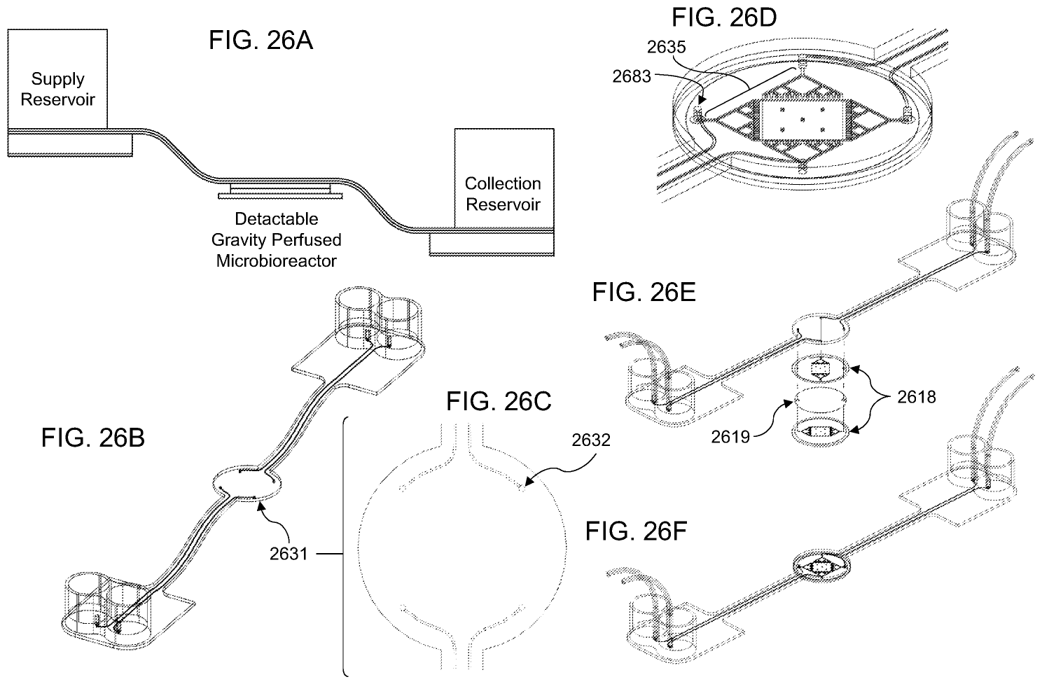

FIGS. 26A-26F describe a versatile, ribbon-based perfusion system that can be used to support a multiplicity of detachable constructs such as two-chamber bioreactor 2635. The basis of this perfusion system consists of pairs of supply reservoirs and collection reservoirs as described elsewhere in this invention, which are connected to universal manifold 2631 by channels within a fluidic ribbon. Universal manifold 2631 serves as a terminal with which bioreactors may be mated. Manifold ports 2632 match with access ports 2683 to complete each circuit through bioreactor 2635.

Bioreactor layers 2618, 2619 may be bonded together or otherwise clamped or affixed to each other, and in turn this assembly may be bonded to manifold 2631, or the whole stack may be clamped together and to manifold 2631, making the bioreactor assembly detachable from the perfusion system.

FIGS. 26A-26B show how the perfusion system may be used in gravity-feed configuration, with supply reservoirs situated at a higher elevation than collection reservoirs. Alternatively, as shown in FIG. 23E-23F, tubing may be inserted into the ports in the reservoir floors such that the system can be actively perfused with pumps. Additionally, the system may be gravity perfused while in a flat, level orientation, in which case the difference in perfusate depth between the supply and collection reservoir would promotes flow.

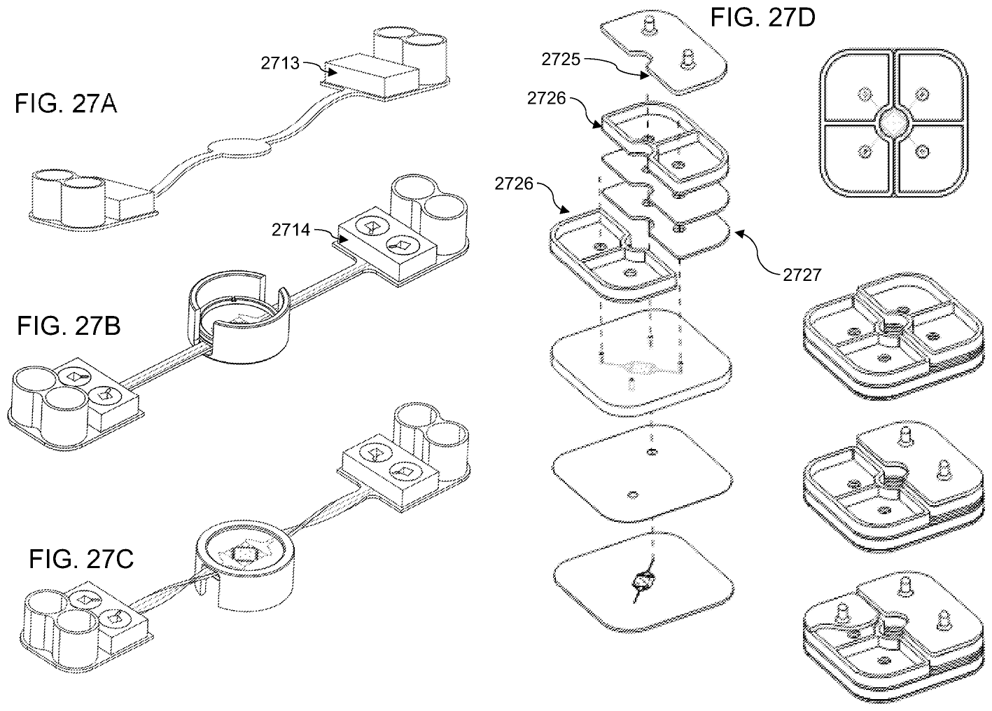

FIG. 27A-27C shows how the twistable ribbon fluidics can be used with either transverse or vertical rotary valve to control flow rate over the default settings specified by the as-fabricated resistances of channels. The ribbon fluidics can be equipped with either a horizontal actuator rotating valves 2713 or a vertical-via rotating valves 2714.

An alternative intermediate between a fixed-design gravity bioreactor as shown in FIGS. 3A-3B and the ribbon fluidics with an adjustable heights in FIGS. 26A-26F is to create general purpose bioreactors and provide user-installed shims to adjust what would otherwise be a fixed height. As shown in FIG. 27D, there are separate bioreactor supply reservoir pairs 2726 and bioreactor collection reservoir pairs 2727. The height of the supply reservoir is adjusted using the appropriate number and thickness of reservoir height adjusting shims 2728 which can bonded in place upon component assembly. The system is competed with reservoir lid with handles 2725.

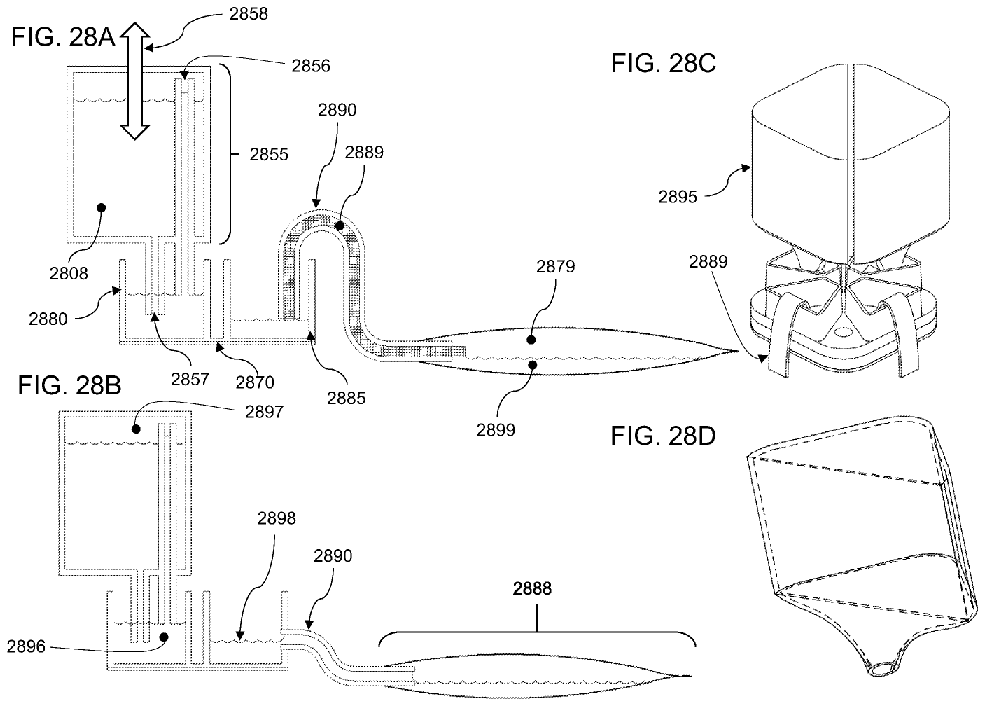

A limitation of the gravity perfusion systems discussed so far is that the level in the supply reservoir drops with time while that in the collection reservoir rises, leading to a steady reduction in the pressure differential between the two sides and a corresponding decrease in flow rate. FIGS. 28A-28D show a self-filling reservoir with adjustable level control and wicking drain for constant pressure head.

The media 2808 in the airtight autofilling supply reservoir 2855 is released only when the fluid level in the bioreactor supply reservoir 2880 drops below the lower end of the level-sensing air vent 2856, at which point air enters autofilling reservoir 2855, increases the volume of air 2897 trapped in autofilling reservoir 2855, and allows fluid to move from the autofilling reservoir 2855 to the bioreactor supply reservoir 2880 through the reservoir delivery tube 2857. Media from the bioreactor supply reservoir 2880 then flows through bioreactor chamber 2870 into bioreactor collection reservoir 2885, increasing the level of media 2898 in that reservoir. An unspecified vertical adjustment means 2858 is used to set the fluid height in the bioreactor supply reservoir 2880. Note that the dual-tube feeder design (2857 and 2856) allows for smaller tubing and a variable length that can allow reservoirs to be remotely located.

On the collection side, the height of fluid in the collection reservoir would normally increase with time and thereby reduce the flow rate through the bioreactor. FIG. 28A shows how this can be avoided by using collection wick 2889, collection tube 2890, and collection bag 2888. The flexibility of the bag can accommodate air 2879 that may pass through the wick.

Alternatively, as shown in FIG. 28C, a siphon tube may be inserted in a side punched hole to adjust output level. This option eliminates the need for a wicking material but may not provide an outlet level of the same precision as the wick.

Together, the large-volume airtight reservoir 2895 maintains a constant supply reservoir height 2895, and the wick or tube collection system ensures that the collection fluid 2896 has a near-constant height so that media 2896 flows from the airtight autofilling reservoir, through the bioreactor supply reservoir, and perfuses the bioreactor at a constant flow rate.

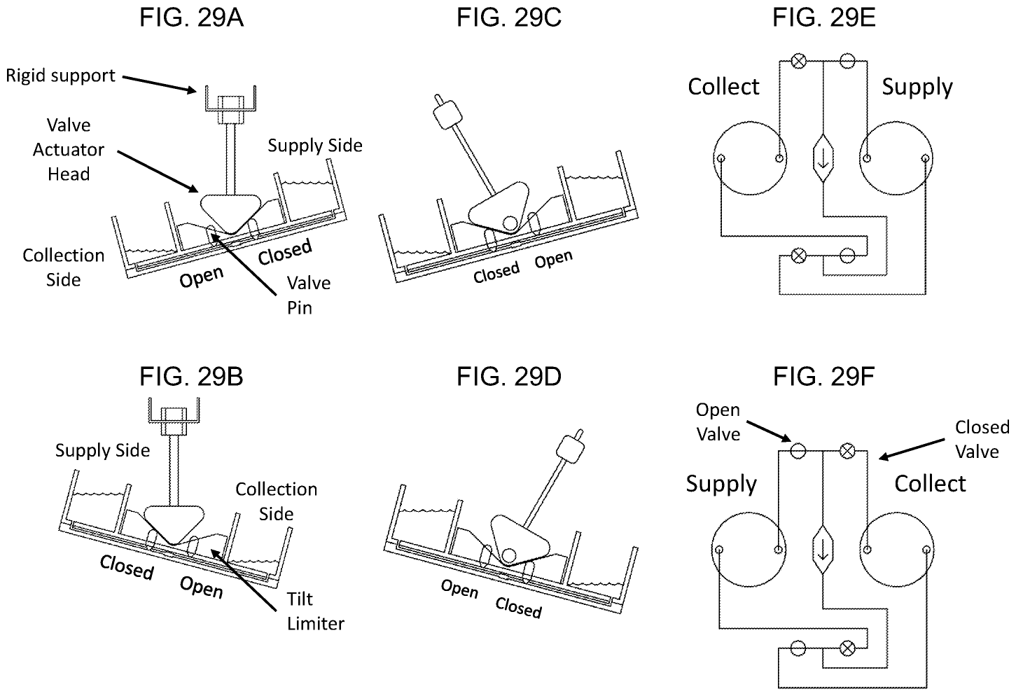

Long-term gravity perfusion of bioreactors is often performed using a tilt table that allows the media to flow back and forth between two reservoirs, in which case the reservoir that is high is the supply reservoir that delivers media to the lower, collection reservoir. When the media in the supply reservoir is depleted, the bioreactor is tilted, so that the functions of the two reservoirs is reversed: supply becomes collection, and collection become supply. The biologically significant limitation of this approach is that the direction of perfusion in the vascular space reverses between the two cycles, and the proper polarization, for example, of the brain microvascular endothelial cells critical for proper performance of the blood-brain barrier is compromised, leading to increased barrier permeability. It is possible to build a fluidic chip that has small, on-board reservoirs and a channel system that supports unidirectional flow without valves. An alternative approach that can support more generalized reservoir and channel design is presented in FIGS. 29A-29D as a tilt-perfused, unidirectional, valved gravity perfusion system. The valve position is controlled by the tilting action of a culture rocker. Rigid support of the valve actuator head enables alignment with the center of the rocker if the entire structure is rigid, as shown in FIGS. 29A-29B. Another embodiment could have a floating support rod that would not require accurate alignment to the center of the rocker, as shown in FIGS. 29C-29D, but would function as an inverted pendulum that was bistable and would quickly move from one side to the other as the system is tilted back and forth. The fluidic schematic governing the two reservoir states is shown in FIG. 29E.

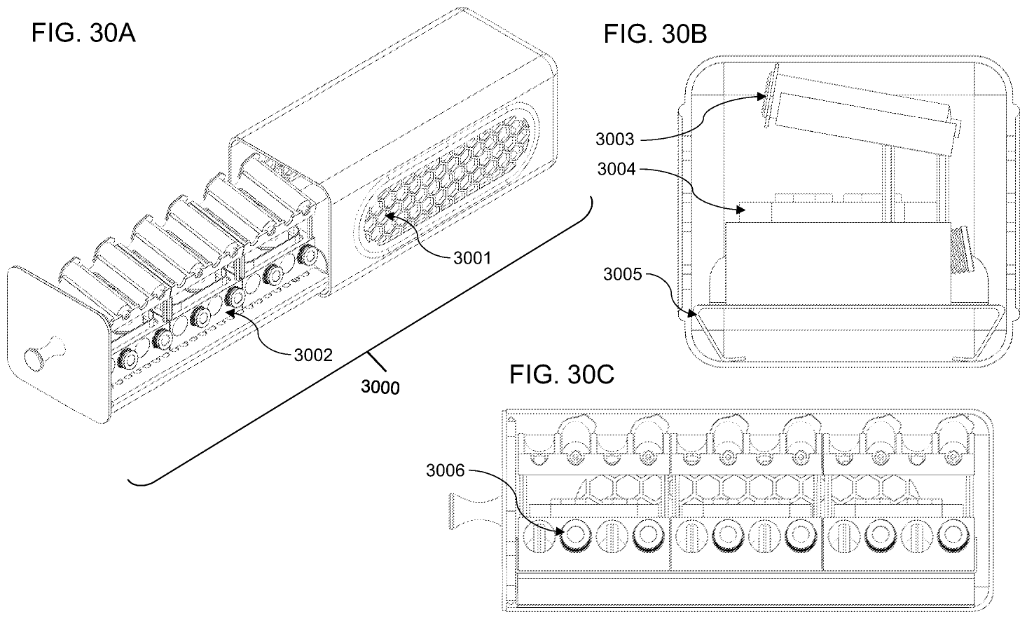

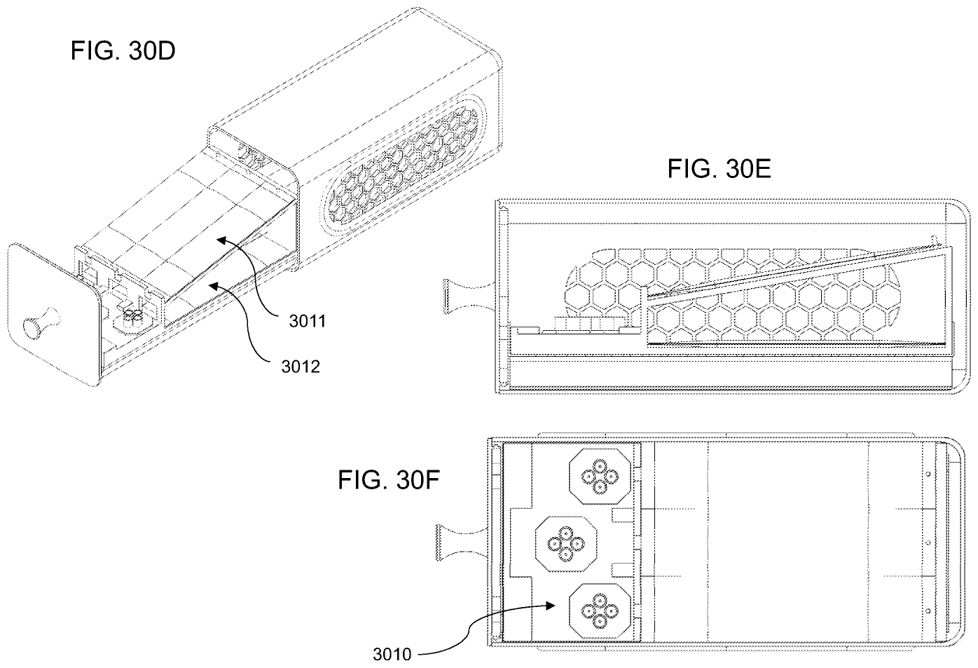

As we have stated earlier, the study of infectious micro-organisms requires not only BSL-3 facilities but also secondary containment. A commercial means for accomplishing this in a standard cell culture environment is to use systems such as the Thermo Fisher Scientific Cell Locker®, a HEPA filtered enclosure for cell culture. FIGS. 30A-30F show how one of these cell lockers can be used to create a system of fully enclosed, tilted reservoirs for long-duration gravity perfusion of multiple independent bioreactors 3000. Embodiments of the gravity perfused bioreactors will allow this locker or functionally similar devices to be used for organ-on-chip studies of microbial infections.

Designed specifically to maximize usable space within the Thermo Fisher Scientific Cell Locker®, the three modules shown in assembly 3000 support cell experiments (each shown with two NVUs resting in Petri dishes or another such containment tray 3004) using gravity and vertical separation to maintain consistent flow to the experiment(s) without requiring electrical power. As shown, these modules provide space for up to 4 standard input syringe vials (with vented plugs) and up to 4 collection vials per module.

The tilted reservoirs provide long-duration gravity perfusion while minimizing the changes in fluid level during the course of the experiment. The HEPA filter window 3001 provides atmosphere exchange between the modules and the incubator in which they are operated. The unit is otherwise sealed to prevent contamination.

The modular array of gravity fed experiments 3002 is supported by sliding drawer/carrier 3005. In this embodiment, each module is shown carrying two experiments within a Petri dish 3004 and fed by the pressure differential caused by the vertical separation of the syringe bodies and the experiments and the experiments to the waste vials 3006 stored underneath. These containers and orientation may vary. Modules shown have optional storage for extra fluid containers both above and below experiments in Petri dish 3004.

The fluid supply reservoir 3003, here represented by a capped and vented syringe body, is angled so as to maximize useful fluid volume while minimizing pressure differential as the fluid drains through the experiment. Fluid flows from supply containers 3003 through tubing (not shown) to the inputs of experiments 3004 and from the experiment outputs through tubing (not shown) to the waste containers 3006.

As an alternative to the vial reservoirs in FIGS. 30A-30C, FIG. 30D-30F shows how the same sliding drawer as 3000 can support bag perfusion and collection reservoirs. The embodiment shown is designed to perfuse three NVUs (shown in spaces 3010 at the front of the drawer using tubing not shown), including tilted supply 3011 bags and waste bags 3012. This layout utilizes gravity to provide near-constant perfusion rates over very long experiments using gas-permeable bags within the same frame 3000.

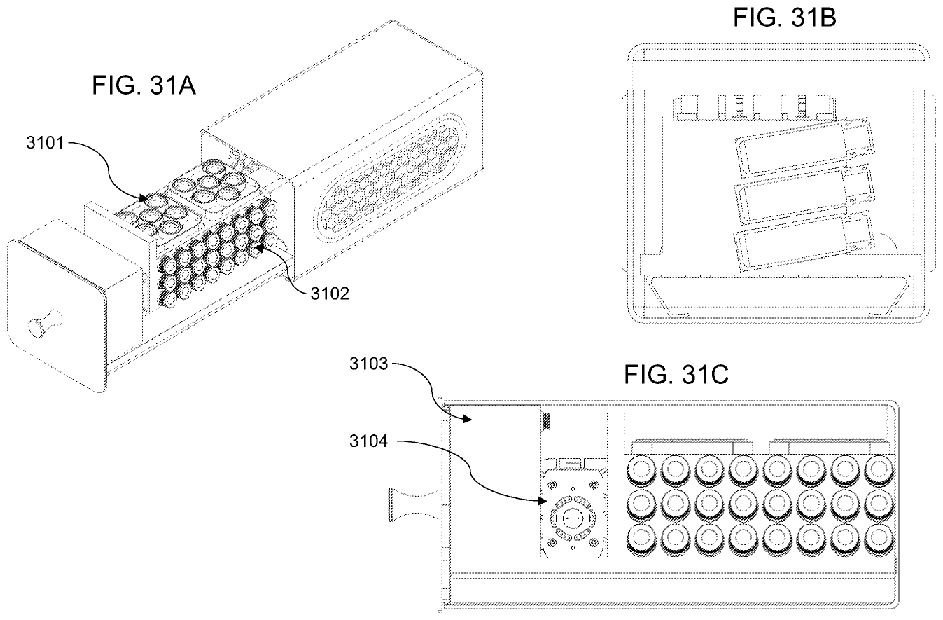

While there are merits to using gravity perfusion, it presents certain limitations, most notably the inability to recirculate media without manually moving media from the collection reservoir back to the supply reservoir, as well as there being no means to performed timed drug, toxin, or microbe delivery without opening the locker. FIGS. 31A-31C show a high-density array of bioreactors with a motorized multi-channel pump running off a sealed gel lead-acid storage battery.

Using the same HEPA filtered storage drawer 3000, this embodiment is focused on maximizing the number of experiments per unit volume and carries a multi-channel pump and battery capable of running the illustrated twelve-experiment set 3101 (with 12 independent fluid channels) continuously. Supply and collection vials 3102 are arrayed beneath the NVU pucks 3101 as shown. Additional images more clearly show the pump 3104 motor and battery unit 3103. Sealed battery chemistry prevents leaks and off-gassing into the cell culture environment. Vials 3102 are vented and tilted such that a long needle is ideally placed to remove as much fluid from the container as possible. Wiring, tubing, and optional digital interface are not shown.

In conclusion, this invention provides detailed descriptions of the four major, integrated subsytems needed for BSL-3 studies of infectious organisms: bioreactors, valves, perfusion reservoirs, and secondary containment.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of the invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LISTING OF REFERENCES

[1] Sung, J H, and Shuler, M L. Prevention of air bubble formation in a microfluidic perfusion cell culture system using a microscale bubble trap. Biomed. Microdevices,
[2] Gao, Y D, Sun, J S, Lin, W H, Webb, D J, and Li, D Y. A compact microfluidic gradient generator using passive pumping. Microfluid. Nanofluid., 12:887-895. 2012.
[3] Markov, D A, Lu, J Q, Samson, P C, Wikswo, J P, and McCawley, L J. Thick-tissue bioreactor as a platform for long-term organotypic culture and drug delivery. Lab Chip, 12:4560-4568. 2012. PMCID: PMC3826880.
[4] Markov, D A, Manuel, S, Shor, L, Opalenik, S R, Wikswo, J P, and Samson, P C. Tape underlayment rotary-node (TURN) valves for simple on-chip microfluidic flow control. Biomed. Microdevices, 12:135-144. 2010.
[5] Weibel, D B, Kruithof, M, Potenta, S, Sia, S K, Lee, A, and Whitesides, G M. Torque-actuated valves for microfluidics. Anal. Chem., 77:4726-4733. 2005.
[6] Brown, J A, Pensabene, V, Markov, D A, Allwardt, V, Neely, M D, Shi, M, Britt, C M, Hoilett, O S, Yang, Q, Brewer, B M, Samson, P C, McCawley, L J M, James M., Webb, D J, Li, D, Bowman, A B, Reiserer, R S, and Wikswo, J P. Recreating blood-brain barrier physiology and structure on chip: A novel neurovascular microfluidic bioreactor. Biomicrofluidics, 9: 054124. 2015. PMCID: PMC4627929.
[7] Brown, J A, Codreanu, S G, Shi, M, Sherrod, S D, Markov, D A, Neely, M D, Britt, C M, Hoilett, O S, Reiserer, R S, Samson, P C, McCawley, L J, Webb, D J, Bowman, A B, McLean, J A, and Wikswo, J P. Metabolic consequences of inflammatory disruption of the blood-brain barrier in an organ-on-chip model of the human neurovascular unit. J. Neuroinflammation, 13: 306. 2016. PMCID: PMC5153753.
[8] Vernetti, L, Gough, A, Baetz, N, Blutt, S, Broughman, J R, Brown, J A, Foulke-Abel, J, Hasan, N, In, J, Kelly, E, Kovbasnjuk, O, Repper, J, Senutovitch, N, Stabb, J, Yeung, C, Zachos, N C, Donowitz, M, Estes, M, Himmelfarb, J, Truskey, G, Wikswo, J P, and Taylor, D L. Functional Coupling of Human Microphysiology Systems: Intestine, Liver, Kidney Proximal Tubule, Blood-Brain Barrier and Skeletal Muscle. Sci. Rep., 7: 42296. 2017. PMCID: PMC5235976.
[9] Brown, J A, Faley, S L, Shi, Y, Hillgren, K M, Sawada, G A, Baker, T K, Wikswo, J P, and Lippmann, E S. Advances in blood-brain barrier modeling in microphysiological systems highlight critical differences in opioid transport due to cortisol exposure. Fluids Barriers CNS, 17: 38. 2020. PMCID: PMC7269003.
[10] Wang, Y I, and Shuler, M L. UniChip enables long-term recirculating unidirectional perfusion with gravity-driven flow for microphysiological systems. Lab Chip, 18:2563-2574. 2018.
[11] Raredon, M. S. B., Ghaedi, M., Calle, E. A., and Niklason, L. E. (2014). A Rotating Bioreactor for Scalable Culture and Differentiation of Respiratory Epithelium. Cell Med 7, 109-121.
[12] Sears, P. R., Yin, W.-N., and Ostrowski, L. E. (2015). Continuous mucociliary transport by primary human airway epithelial cells in vitro. Am. J. Physiol. Lung Cell Mol. Physiol. 309, L99-108.
[13] E. Berthiera and D. J. Beebe “Flow rate analysis of a surface tension driven passive micropump” Lab Chip, 2007, 7, 1475-1478.
[14] Takayama Braille: “Computerized microfluidic cell culture using elastomeric channels and Braille displays”, PNAS Nov. 9, 2004 101 (45) 15861-15866.
[15] X Wang, C Cheng, S Wang, and Sh Liu “Electroosmotic pumps and their applications in microfluidic systems” Microfluid Nanofluid (2009) 6:145-162.
[16] S K. Vajandar, D Xu, J Sun, D A. Markov, W H. Hofmeister, and D Li, “Field-Effect Control of Electroosmotic Pumping Using Porous Silicon—Silicon Nitride Membranes” J Microelectromechanical Systems, Vol. 18, No. 6, 1173-1183.
[17] B D. Iverson, S V. Garimella “Recent advances in microscale pumping technologies: a review and evaluation” Microfluid Nanofluid (2008) 5:145-174.
[18] D J Laser and J G Santiago “A review of micropumps” Micromech. Microeng. 14 (2004) R35-R64. W Zhang, S Lin, Ch Wang, J Hu, C Li, Zh Zhuang, Y Zhou, R A. Mathies and Ch J Yang “PMMA/PDMS valves and pumps for disposable microfluidics” Lab Chip, 2009, 9, 3088-3094.
[19] W. H. Grover, A. M. Skelley, C. N. Liu, E. T. Lagally, R. A. Mathies, Sens. Act. B. 89, 315 (2003).
[20] J Kim, D Chen and H H. Bau, “An automated, pre-programmed, multiplexed, hydraulic microvalve” Lab Chip, 2009, 9, 3594-3598. D. A. Markov, S. Manuel, L. Shor, S. Opalenik, J. Wikswo, and P. Samson, “Tape Underlayment Rotary-Node (TURN) Valves for Simple On-Chip Microfluidic Flow Control”, Biomedical Microdevices 12, 135-144, 2010.
[21] Marie-Elena Brett et al, “Controlling flow in microfluidic channels with a manually actuated pin valve”, Biomed Microdevices (2011) 13:633-639.
[22] Hulme, S. E., et al. (2009). “Incorporation of prefabricated screw, pneumatic, and solenoid valves into microfluidic devices.” Lab on a Chip 9(1): 79-86.
[23] R Johann and P Renaud “A simple mechanism for reliable particle sorting in a microdevice with combined electroosmotic and pressure-driven flow” Electrophoresis 2004, 25, 3720-3729.
[24] T Pan, S J McDonald, E M Kailand B Ziaie “A magnetically driven PDMS micropump with ball check-valves” J. Micromech. Microeng. 15 (2005) 1021-1026.

1. A vertical-via rotary valve (VVRV), comprising:
a valve body having a housing;
one or more fluidic channels, each fluidic channel having a vertical channel portion defined in the valve body and being adjacent to the housing, wherein a fluid flow through the vertical channel portion is controllable by deforming a sidewall of the vertical channel portion; and
an actuator received in the housing and rotatably engaged with the one or more fluidic channels to operably control the fluid flow through the vertical channel portion of each fluid channel, wherein the actuator comprises:
one or more actuating pins accommodated in a pin housing and corresponding to the vertical channel portions of the one or more fluidic channels, wherein each actuating pin is movable from a first position in the pin housing to a second position out of the pin housing when pushed, otherwise, said actuating pin returns from the second position to the first position, wherein when said actuating pin is in the first position, it un-pinches a corresponding vertical channel portion so that the fluid flow therethrough is not restricted, and when said actuating pin is in the second position, it pinches the corresponding vertical channel portion so that the fluid flow therethrough is restricted;
a pin actuator cam having at least one lobe; and
a driving shaft engaged with the pin actuator cam to operably rotate the pin actuator cam, such that when the lobe is aligned with an actuating pin, the lobe pushes said actuating pin to move from the first position to the second positon, thereby restricting the fluid flow through said corresponding vertical channel portion, when the lobe is misaligned with said actuating pin, said actuating pin returns from the second position to the first position, thereby, unrestricting the fluid flow through said corresponding vertical channel portion.

2. The VVRV of claim 1, wherein the valve body is formed of an elastomeric material including polydimethylsiloxane (PDMS).

3. The VVRV of claim 1, wherein the vertical channel portion of each fluidic channel is a vertical via.

4. The VVRV of claim 3, wherein the actuator features a vertical trough aligned with the vertical via, such that when the actuator un-pinches the vertical via and the fluid flow through the vertical via is allowed, and when the actuator rotates from the un-pinched position, the vertical via is pinched closed and the fluid flow is restricted.

5. The VVRV of claim 1, wherein the vertical channel portions of the one or more fluidic channels in the valve body are formed in a single layer or multiple layers in the valve body.

6. The VVRV of claim 1, wherein the vertical channel portion of each fluidic channel is in fluid communication with a first port and a second port.

7. The VVRV of claim 1, wherein the actuator comprises one or more rolling members, and a driving member for driving the one or more rolling members to rotate independently and/or coordinately, such that when actuated, the one or more rolling members selectively address the vertical channel portions of the one or more fluidic channels so as to control rates of the fluid flow through the vertical channel portions of the one or more fluidic channels independently and/or coordinately.

8. The VVRV of claim 7, wherein the one or more rolling members comprise one or more actuating discs, wherein each actuating disc is formed with a tapered or raised protrusion such that rotation of said actuating disc results in the vertical channel portion of a corresponding fluidic channel in a fully closed state, a partially closed state, or a fully open state, depending upon a position of said tapered or raised protrusion relative to the vertical channel portion.

9. The VVRV of claim 8, wherein the one or more actuating discs are concentrically stacked to one another.

10. The VVRV of claim 8, wherein the driving member comprises one or more nesting wrenches.

11. The VVRV of claim 7, wherein the one or more rolling members comprise a plurality of rollers or a plurality of planetary gears rotatably engaged with the driving member.

12. The VVRV of claim 11, wherein the driving member comprises a top plate, a bottom plate and a central hub connected to central portions of the top plate and the bottom plate, and wherein the plurality of rollers is rotatably affixed to edge portions of the top plate and the bottom plate, such that rotation of the central hub rotates causes each roller to rotate around its own axis but not translate relative to the top plate and the bottom plate.

13. The VVRV of claim 11, wherein the driving member comprises a top plate, a bottom plate, a driving shaft connected to central portions of the top plate and the bottom plate, and a sun gear affixed to the driving shaft and situated between the top plate and the bottom plate, wherein each planetary gear is meshed with the sun gear and rotatably affixed between the top plate and the bottom plate, such that the rotation of the drive shaft is transferred to the sun gear, which in turn causes each planetary gear to rotate around its own axis but not translate relative to the top plate and the bottom plate.

14. (canceled)

15. The VVRV of claim 1, wherein each actuating pin is accommodated in the pin housing with an elastic member including a spring.

16. The VVRV of claim 1, wherein the return force on the actuator pin is provided by the elastomeric material of the valve body, including PDMS.

17. The VVRV of claim 1, being a mixing valve.

18. The VVRV of claim 1, wherein the valve body is disposable, while the actuator that is never in contact with fluid is reusable.

19-63. (canceled)
